(19) United States
(12) Patent Application Publication
(10) Pub. No.: US 2023/0264200 A1
(43) Pub. Date: Aug. 24, 2023

(54) AN AUTOMATED QUANTITATIVE ASSAY DEVICE AND A METHOD OF PERFORMING THE QUANTITATIVE ASSAYS

(71) Applicant: LANARKSHIRE GLOBAL LLC, Chester Springs, PA (US)

(72) Inventors: DINO ROTONDO, Lancashire (GB); WILLIAM STIMSON, Scotland (GB); DAVID COWAN, Scotland (GB)

(21) Appl. No.: 18/003,163

(22) PCT Filed: Jun. 26, 2021
(86) PCT No.: PCT/IB2021/055726

§ 371 Date: Dec. 23, 2022

(30) Foreign Application Priority Data
Jun. 26, 2020 (GB) 2009823.2

(51) Int. Cl. B01L 3/00; G01N 33/543; G01N 35/00

(52) U.S. Cl. CPC B01L 3/50851; B01L 3/50853; G01N 33/54366; G01N 35/00584; B01L 2200/0647; B01L 2200/04; B01L 2200/0689; B01L 2200/16; B01L 2300/044; B01L 2300/047; B01L 2300/0663; B01L 2300/0861; B01L 2300/18; B01L 2400/043; G01N 2035/00019; G01N 2035/00346

(57) ABSTRACT

The present invention relates to the field of assay devices and methods for performing assays, such as immunoassays. The system comprises a means to receive the target sample collected from a subject; a means to measure the target analyte possibly present in said target sample; and a process of measuring the target analytes in real time manner. The target analyte includes but is not limited to any biological analyte, microbial entity like those of viral or bacterial sources such as SARS-CoV-2. The invention thus primarily relates to measuring analytes of interest to detect and treat related indications, such as COVID-19.

### FIELD OF THE INVENTION

The present invention generally relates to the field of assay devices and method for performing assays, such as immunoassays. In particular, the present invention relates to an automated quantitative assay device and a method of performing heterogenous assay suitable for point of care/use applications.

### BACKGROUND OF THE INVENTION

An assay is an analytical procedure for qualitatively or quantitatively assessing the presence, amount or the functional activity of a target entity (known as an analyte) in a sample, such as a biological sample. Assays are common laboratory procedures in the medical, pharmacological, environmental/molecular biological fields, used to detect analytes such as drug compounds, biochemical substances or particular cell types.

Many assays, particularly those used to make quantitative measurement of an analyte are complex procedures, which must be performed by skilled personnel. As such, they can be time consuming and expensive to conduct, and samples to be assayed are often sent away to dedicated laboratory facilities with results available days or weeks later. There is always an associated risk of mishandling, like non maintenance of temperature during transit, agglutination of samples, mixing of samples and sometimes breach of threshold time between collection of sample and actual testing.

“Point of care” diagnostic tests (or, for non-clinical applications, “point of use” testing) can convey certain benefits over laboratory tests. Point-of-care (POC) is one of the largest growing trend for the diagnosis and cure of a disease. The terms “point of care” and “point of use” are generally understood to include diagnostic tests/assays in which samples, such as patient specimens, are assayed at or near the sampling location such that completion of the assay and any follow-up action based on the results can be completed within the same patient/problem encounter. The Point of Care (POC) generally includes the assay which are performed on a sample or specimen extracted from patient's body and tested near the sampling location so that any follow up action, i.e. any treatment if required after diagnosing the patient's sample or specimen could be done on the spot without any such delay. POC devices are widely used to rapidly and easily diagnose a disease as well as provide cure or prevention to fight against a disease or infection.

Medical/veterinary applications of point of care testing include intensive/emergency care encounters, tests conducted in hospital wards, general practice, outpatient clinics, veterinary surgeries, and the like. Non-medical applications may be found in workplaces and homes. Point of use (i.e. nonclinical) applications include quality control testing, for example in the manufacture and packaging of food or pharmaceuticals, domestic, chemicals, or testing of water quality.

All these applications share the common requirement of a rapid turnaround and communication of results to guide decisions. In addition, point of use/care assays must be comparatively simple to perform, yet yield robust and reliable results. This combination of features is difficult to achieve and, as a consequence, such assays may provide only qualitative or semi-quantitative results. For example, many simple lateral flow type tests for pregnancy or stigmatized diseases such as HIV may provide a coarse “yes” or “no” result, to provide an indication as to whether further laboratory testing is required. However, the results cannot in themselves inform clinical decisions.

The capability of conducting such competitive or sandwich assays in a point of care/use setting is in principle very desirable. Diagnosis without the need for samples to be referred to centralised testing laboratories can be particularly important in resource-limited settings, such as medical encounters in remote locations. The elimination of lengthy delays also has the potential to improve clinical outcomes more generally, by preventing patients from leaving a treatment conduit during the delay before results are available.

The requirement for an assay to be robust and reliable, and sufficiently simple to be performed in a point of use/care setting presents a significant barrier to implementation, and many types of assays are considered to be unsuitable for any type of point of care/use application. Efforts have been made to design assays which can be performed in a single step, by mixing reagents in a “one pot reaction” (also known as a “homogeneous” assay). However, even when this has been possible, the quality of data obtainable is comparatively poor. For example, enzyme-linked immunoassays (ELISAs) are sensitive to “noise” caused by components of the sample, or matrix. In order to obtain quantitative or semi-quantitative results, such assays must therefore include a complex series of reagent and rinse solution washing steps in order to remove the unwanted components. So called “matrix effects”, which are a barrier to point-of-care applications are described for example by Chiu et al., Journal of Laboratory Automation, June 2010, 233-242. The influence of matrix effects on the quality of data and assay efficiency is also described for example by Saab et al., International Journal of High throughput Screening, 2010:1, 81-98, and Imbert, P. E. et.al., Assay Drug Dev Technol., 2007, June; 5 (3):363-72.

Nevertheless, U.S. Pat. No. 11/908,071 discloses a dual path immunoassay device, wherein the invention include test cells with a first sorbent material defining a first flow path for a solution, a second sorbent material defining a second flow path distinct from the first flow path for a sample, and a test site with immobilized antigens or antibodies or other ligand binding molecules such as aptamers, nucleic acids, etc. located at the junction of the first and second sorbent materials for identifying one or more ligands. The first and second sorbent strips touch each other at the test site location. The test has a disadvantage of being qualitative chromatographic assay.

US20160195524A1 discloses an automated system for performing a heterogeneous assay comprising of an assay cassette for use in performing a heterogeneous assay, the assay cassette comprising of a fluid conduit and one or more chambers in the fluid conduit, from which a measurement may be acquired from a sample using a cassette reader device. Also disclosed is a tablet or bead for use with the assay system, which may be incorporated in the cassette. The tablet or bead may comprise one or more reagents to be used in the assay, in a soluble matrix. Use of an acridan or acridinium ester label may enable a sensitive measurement to be rapidly acquired. The assay may be configured to be performed by a clinician at the point of use or care.

US2013/0143328A1 discloses an automated assay fluid dispensing system includes a database that associates assay protocols with assay procedures, the procedures including a first assay procedure specifying dissimilar first and second channel procedures for driving first and second channels of a fluid-dispenser cassette.

U.S. Pat. No. 14/427,880 discloses a point-of-care lateral flow immunochromatographic assay for direct detection of enteroviruses. In particular, the present disclosure relates to a point-of-care lateral flow immunochromatographic assay for detection of the etiologic agents of Hand, Foot and Mouth Disease (HFMD), using antibodies specific for enteroviruses.

Simplifying such assays sufficiently to enable them to be performed at the point of care/use without the need for specialist training has therefore remained a challenge. Accordingly, there remains a need for improved methods and apparatus for performing heterogeneous assays. In view of the cited prior art, the present invention aims to overcome the drawbacks of the prior art as well as to provide a quick, heterogeneous, simple and quantitative immunoassay device and method for performing immunoassay at point of care/use. This particular patent application is non-limiting to measurements of analytes associated with the COVID 19 outbreak.

### OBJECT OF THE INVENTION

Accordingly, the primary object of the invention is to provide a quick, heterogeneous, simple and quantitative immunoassay device.

Another object of the invention is to provide a method for performing heterogeneous and quantitative immunoassay.

Another object of the invention is to provide automated quantitative assay device suitable for point of care/use applications.

Another object is to provide an automated immunoassay device and method for performing immunoassay at point of care/use.

Yet another object of the invention is to provide an automated immunoassay device and method for performing immunoassay for measurement of analytes associated but not limited to SARS-CoV2 or viral or bacterial outbreak at point of care/use.

### SUMMARY OF THE INVENTION

The present invention generally relates to the field of assay devices and method for performing assays, such as immunoassays. In particular, the present invention relates to an automated quantitative assay device and a method of performing for performing heterogeneous assay suitable for point of care/use applications.

In an aspect of the present invention, an automated device for measuring at least one target analyte is proposed. The system comprises a means to receive the target sample collected from a subject; a means to measure the target analyte possibly present in said target sample; and a process of measuring the target analytes in real time manner. The target analyte includes but is not limited to any biological analyte, microbial entity like those of viral or bacterial sources such as SARS-CoV-2. The invention thus primarily relates to measuring analytes of interest to detect and treat related indications, such as COVID-19. The measuring process includes but is not limited to a competitive or sandwich assays, immunological assays etc. The means to receive the target sample comprises a cassette. The device comprises the cassette, the means or instrument for measuring the analyte and the chemistry of the detection process. The whole device is designed so that sample may be collected in a standard way and injected directly into the cassette, the cassette is then inserted into the instrument, the measurement performed and the result is then reported.

In another aspect, the present invention proposes a method for automated quantitative assay samples containing at least one analyte by injecting the sample in a cassette fully loaded with beads and antibody/conjugate cocktail. The cassette is then inserted into the device for measuring the target analyte quantitatively by the use of measuring instrument. Thereafter the magnetic stirrer is activated to mix the sample and the conjugate/antibody cocktail. A neutral liquid or air is then pumped into the mixing chamber causing the sample/conjugate/antibody mixture to be displaced and to flow round the fluid conduit immersing the beads. The liquid is then heated to approximately 37° C. to allow incubation and pumping the wash through fluid conduit to remove all of the remaining sample not bound to the beads. The wash cycles are repeated and fluid conduit of liquid is purged by pumping air through the conduit between the wash cycles. Chemiluminescence is measured through sensors and result recorded. Thereafter the cassette is ejected from the measuring instrument.

These and other features and advantages of the various embodiments will appear more fully from the following description and the accompanying drawings, which are incorporated in and constitute a part of the specification.

### BRIEF DESCRIPTION OF DRAWING

The invention will now be described by way of following reference drawings as exemplary embodiments of the invention:

FIG. 1 shows schematics of the measuring instrument with cassette in accordance with a preferred embodiment of the present invention.

FIGS. 2(a), 2(b) and 2(c) show the configuration of cassette in accordance with a preferred embodiment of the present invention.

FIG. 2(d) gives the box with the top cover cut out with a number of the items above marked according to the present invention.

FIG. 2(e) shows the diagram of measuring instrument along with the attachments to the cassette device in accordance with the present invention.

FIG. 3 shows detailed measurement part of the system according to the present invention.

FIG. 4 shows Silicon Photomultiplier type sensor according to the present invention.

FIG. 5 shows the graphical representation of ELISA Inter Assay calibration line for TSH Assay according to the present invention.

FIG. 6 shows the graphical representation of Bead Plate Inter Assay calibration line for TSH Assay according to the present invention.

FIG. 7 shows the graphical representation of Quantilyte Inter Assay calibration line for TSH Assay according to the present invention.

FIG. 8 represents graphically curve comparison using ELISA, Bead plate Interassay and Quantilyte Inter assay for TSH Assay according to the present invention.

FIG. 9 shows the comparison graphically using ELISA, Bead plate Interassay and Quantilyte Inter assay for 50 μl U/ml quality control samples of TSH Assay at according to the present invention.

FIG. 10 shows the comparison graphically using ELISA, Bead plate Interassay and Quantilyte Inter assay for 300 μl U/ml quality control samples of TSH Assay at according to the present invention.

FIG. 11 shows the comparison graphically using ELISA, Bead plate Interassay and Quantilyte Inter assay for 1800 μl U/ml quality control samples of TSH Assay at according to the present invention.

FIG. 12 graphically shows the % BIAS values for ELISA, Bead plate Interassay and Quantilyte at 50 μl U/ml, 300 μl U/ml and 1800 μl U/ml QC levels of TSH assay according to the present invention.

FIG. 13 graphically represents % CV values for ELISA, Bead plate Interassay and Quantilyte at 50 μl U/ml, 300 μl U/ml and 1800 μl U/ml QC levels of TSH Assay according to the present invention.

FIG. 14 shows bar diagrams comparing the results for lyphocheck level 3 using different methods according to the present invention.

FIG. 15 shows TT4 standard curve according to the present invention.

FIG. 16 shows Quantilyte TT4 standard curve according to the present invention.

FIG. 17 shows cortisol standard curve according to the present invention.

FIG. 18 shows Quantilyte cortisol standard curve according to the present invention.

FIG. 19 shows Lyphocheck QC's ELISA and Quantilyte results for TT4 assay according to the present invention.

FIG. 20 graphically represents % CV values for Quantilyte vs ELISA assays for TT4 assay according to the present invention.

FIG. 21 shows Lyphocheck QC's ELISA and Quantilyte results for cortisol assay according to the present invention.

FIG. 22 graphically represents % CV values for Quantilyte vs ELISA assays for cortisol assay according to the present invention.

FIG. 23 graphically represents Cat sample ELISA vs Quantilyte results for TT4 assay according to the present invention.

FIG. 24 graphically represents Cat sample ELISA vs Quantilyte % difference plot for TT4 assay according to the present invention.

FIG. 25 shows graphically Cat sample ELISA vs Quantilyte results for cortisol assay according to the present invention.

FIG. 26 graphically represents Cat sample ELISA vs Quantilyte % difference plot for cortisol assay according to the present invention.

FIG. 27 shows % Maximum Signal for CRP luminescent values according to the present invention. and OD of calibration lines.

FIG. 28 shows % OD for CRP sample curve according to the present invention.

### DESCRIPTION OF THE INVENTION

The present invention is directed toward assay devices for detection of one or more analytes in a sample. The assay devices are constructed in a manner to allow for the real-time interaction of the assay reagents. In a preferred embodiment of the present invention, a device for measuring at least one target analyte is proposed. The device comprises a means to receive the target sample collected from a subject; a means to measure the target analyte possibly present in said target sample; and a process of measuring the target analytes in real time manner. The target analyte includes but is not limited to any biological analyte, microbial entity like those of viral or bacterial sources such as SARS-CoV-2. The invention thus primarily relates to measuring analytes of interest to detect and treat related indications, such as COVID-19. The measuring process includes but is not limited to a competitive or sandwich assays, immunological assays etc. The means to receive the target sample comprises a cassette. The system comprises the cassette, the means or instrument for measuring the analyte and the chemistry of the detection process. The whole system is designed so that body fluid may be collected in a standard way and injected directly into the cassette, the cassette is then inserted into the instrument, the measurement performed and the result is then reported. Further included in this invention is a method for performing the immunoassay.

As will be understood by the ordinarily skilled artisan upon reading the specification, the analyte can be any specific substance or component that one is desirous of detecting and/or measuring in a chemical, physical, enzymatic, or optical analysis. Analytes of interest include, for example, novel coronavirus 2019-nCoV, SARS-CoV2 or viral or bacterial outbreak, antigens (such as antigens specific to bacterial, viral or protozoan organisms); antibodies, particularly those induced in response to an infection, allergic reaction, or vaccine; hormones, proteins and other physiological substances (for example, human chorionic gonadotropin, estrogens, progestins, testosterones, corticosteroids, human growth factors, hemoglobin, and cholesterol); nucleic acids; a variety of enzymes; therapeutic compounds and illicit drugs; contaminants and environmental pollutants; or any number of natural or synthetic substances.

As is appreciated by one skilled in the art, the number of natural and synthetic substances which can be detected by the assay devices and methods of the present invention is extensive, and include, but is not limited to, the following: ACE inhibitors, adrenergics and anti-adrenergics, alcohol deterrents (for example, disulfiram), anti-allergics, anti-anginals, anti-arthritics, anti-infectives (including but not limited to antibacterials, antibiotics, antifungals, antihelminthics, antimalarials and antiviral agents), analgesics and analgesic combinations, local and systemic anesthetics, appetite suppressants, antioxidants, anxiolytics, anorexics, antiarthritics, anti-asthmatic agents, anticoagulants, anticonvulsants, antidiabetic agents, antidiarrheals, anti-emetics, anti-epileptics, antihistamines, anti-inflammatory agents, antihypertensives, antimigraines, antinauseants, antineoplastics, antioxidants, antiparkinsonism drugs, antipruritics, antipyretics, antirheumatics, antispasmodics, antitussives, adrenergic receptor agonists and antagonists, anorexics, appetite suppressants, cardiovascular preparations (including anti-arrhythmic agents, cardiotonics, cardiac depressants, calcium channel blockers and beta blockers), cholinergics and anticholinergics, contraceptives, diuretics, decongestants, growth stimulants, herbal preparations, hypnotics, immunizing agents, immunomodulators, immunosuppresives, muscle relaxants, neurologically-active agents including anti-anxiety preparations, antidepressants, antipsycotics, psychostimulants, sedatives and tranquilizers, sore throat medicaments, sympathomimetics, vasodilators, vasoconstrictors, vitamins, xanthine derivatives, various combinations of these compounds, and the like.

As used herein, the term “reagent’ is used to indicate any liquid, e.g., a solvent or chemical solution which is to be mixed with a sample and/or other reagent in order, e.g., for a reaction to occur or to enable detection. A reagent can be, for example, another sample interacting with a first sample. A reagent can also be a diluting liquid such as, e.g., water. A reagent may comprise an organic solvent or a detergent. A reagent may also be a buffer. A reagent in the stricter sense of the term may be a liquid solution containing a reactant, typically a compound or agent capable, e.g., of binding to or transforming one or more analytes present in a sample. Examples of predefined reactants are, for example, and not limited thereto, enzymes, enzyme substrates, protein reagents, chemical reagents, sera reagents, conjugated dyes, protein-binding molecules, nucleic acid binding molecules, antibodies, chelating agents, promoters, inhibitors, epitopes, antigens, catalysts, etc.

Optionally, dry reagents may be present in the analytical device and be dissolved by a sample, another reagent, or a diluting liquid.

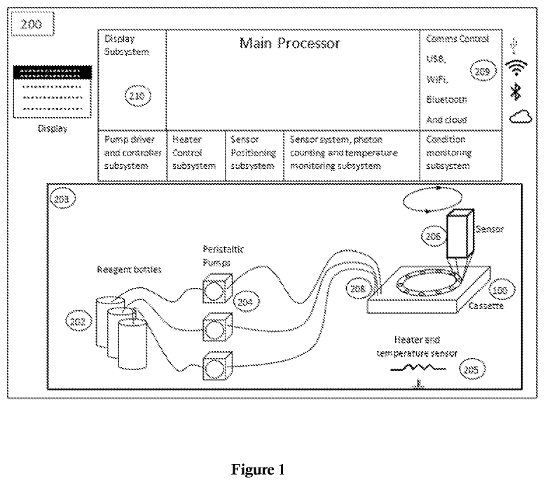

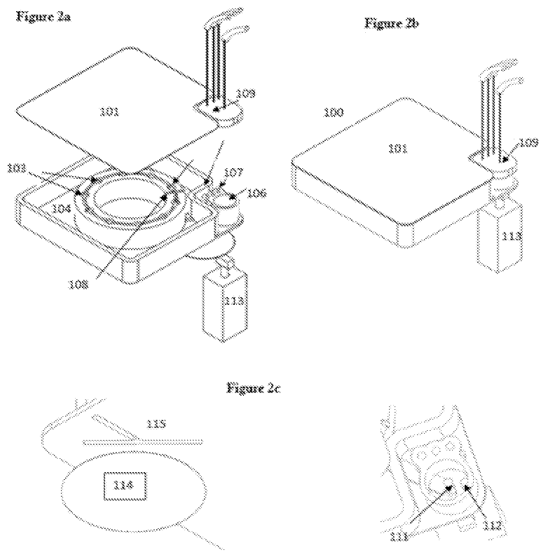

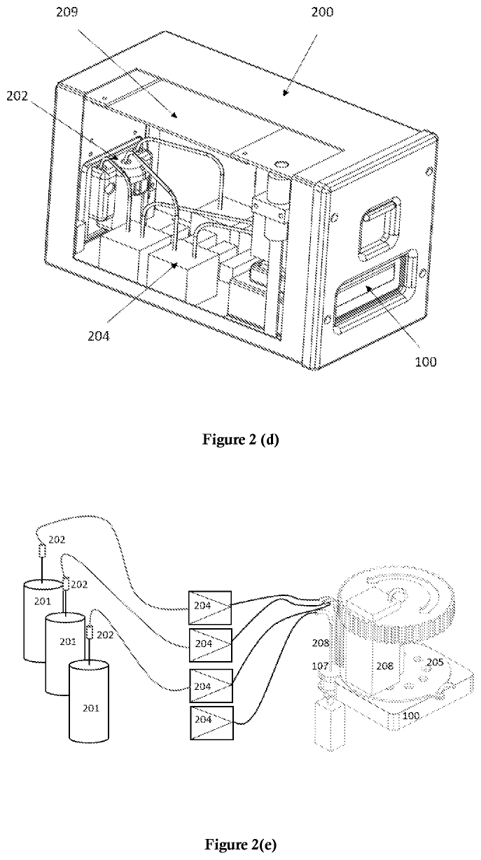

In an embodiment of the present invention, FIG. 1 represents a schematic presentation of measuring instrument 200 and FIG. 2(d) shows the diagram of measuring instrument in accordance with the present invention. The instrument is a closed housing 203 to prevent the enclosure from external light. The housing contains a plurality of reservoirs 201 containing predefined reagents inserted into enclosure connected with injection needles 202 which are connected to pump 204. Peristaltic or Syringe Pumps 204 that help to pump the reagents to the inlet ports 107, present at the top of the cassette. through injection needles 208. The cassette 100 receives the reagents and heating element 205 along with injection needles 208 are moved as a unit to engage with the cassette. The photo sensor 206 scans the cassette for detecting chemiluminescence. The measuring instrument has an automated opening to allow the cassette device to be inserted, an electronic controller 209 to communicate with the external devices via wi-fi or bluetooth or wired communication. The measuring instrument will communicate with the outside world by passing commands and results over wired and wireless communications links such as Bluetooth, WiFi, USB and directly to the cloud using the latest mobile communication protocol such as 4G or 5G 210. Alternatively, the results may be displayed at the instrument on an LCD display or a mobile phone or tablet incorporated into the instrument 210.

The automated quantitative assay device is capable of performing heterogeneous competitive or sandwich assays or immunological assays of a target sample across an elevated range in 10-15 minutes.

In another embodiment, FIGS. 2(a), 2(b) and 2(c) show the cassette device 100 having a sample inlet 105 which opens into a fluid conduit 102 containing beads 103 fitted into pockets in conduit so that beads do not move with fluid. The extra fluid gets collected in waste reservoir 104 through outlet 108. A sample port opens in the mixing chamber 106 which is connected to a reagent port 107. The fluid conduit is a continued channel fluidly connecting the inlet port, mixing chamber and the reagent port. The cassette has a transparent cover 101 to allows recording of chemiluminescence. The mixing chamber is enclosed with a silicone rubber cap 109 which can be pierced with the injection needles.

In this particular implementation there are channels shown on the rear of the cassette used to transport fluids from the mixing chamber 106 and reagent port 107. These channels 115 are sealed by an adhesive patch 114 shown in FIG. 2(c).

In another embodiment of the present invention, the beads 103 are pre-labelled immobilized analyte-specific probe are placed individually in a plurality of wells of the cassette device which is sealed using permanent heat seals.

In yet another embodiment of the present invention, the cocktail of conjugate molecules may be pre-packaged in the cassette device in solid form 112 (FIG. 2(c)), formulated to dissolve to form a reagent fluid.

In another embodiment, the present invention provides an automated quantitative assay device having a mixing chamber which is filled with a predetermined amount of a cocktail of conjugate molecules tagged with a chemiluminescent molecule, facilitating a competitive assay measurement wherein said conjugate molecules in the cocktail has an individual predetermined concentration.

In still another embodiment of the present invention, the mixing chamber is filled with a predetermined amount of the cocktail of conjugate molecules tagged with a chemical label that binds specifically to the analyte specific probes tagged with the chemiluminescent molecule, facilitating a sandwich assay measurement wherein each analyte specific probe in the cocktail of conjugate molecules have an individual predetermined concentration.

In another embodiment, the present invention provides a mixture of the 2 implementations, viz. a cocktail of conjugate molecules tagged with a chemiluminescent molecule, and a cocktail of analyte specific probe molecules tagged with a chemiluminescent, are possible in the same mixing chamber. At least two chambers may comprise the same type of immobilized analyte-specific probe. Thus, the cassette may be configured for an assay and a confirmatory assay to be conducted. A transparent layer is used to contain the fluid conduit, the beads (used for immobilizing the analyte specific molecule), the mixing chamber and the waste reservoir. The beads, and the antibody cocktail are inserted before the transparent layer is fixed.

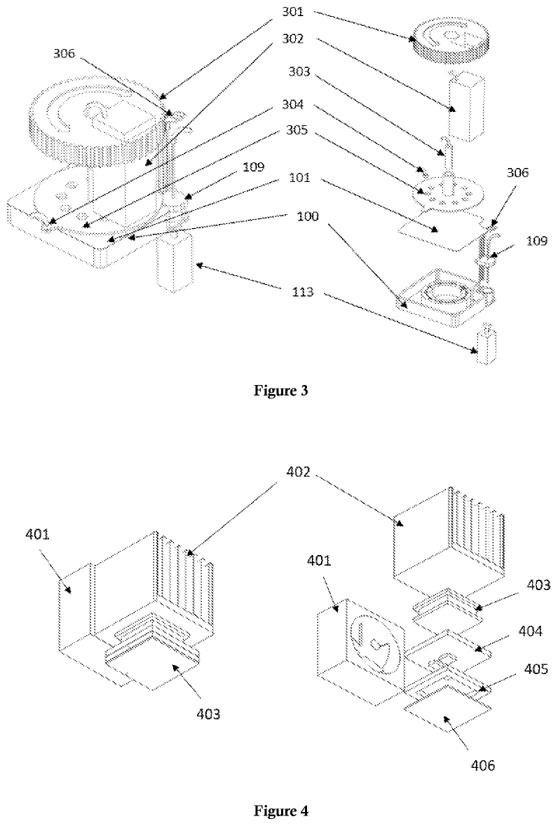

In another embodiment FIG. 3 shows the measurement instrument of the device in more detail. The main gear 301 is driven by either a stepper or geared DC motor and is used to locate the sensor 206 above each of the measurement locations. The cartridge heater 304 and associated heating element 205 is used to heat the temperature of which is set at 37° C. during the heating process. The temperature is monitored by a temperature sensor 304. The injection needles 208 are used to inject reagents into the cassette 100 through the silicone rubber cap 109.

In another embodiment, a small magnet 111 may be placed in the mixing chamber to facilitate magnetic stirring. A motor 113 with a drive magnet attached to its shaft. When the shaft and magnet is rotated by the motor the small magnet in the mixing chamber 111 is caused to rotate causing the contents of the chamber to mix thoroughly.

Once the cassette is inserted, the closure of the insertion slot application of the heating element and the insertion of the injection needles into the cassette is caused by one single action. This may either be done by the machine when the measurement is initiated or manually by the user. At the start of each measurement the heating element is caused to move upwards to make contact with the high thermal conductivity material deposited on the bottom of the cassette, or alternatively a heating element with apertures to allow light to pass will move downwards to press on the top of the cassette. At the same time the injection needles move upwards piercing the rubberized cap 109 forming the seal to the inlet ports.

In still another embodiment, the present invention provides a heating element will be held, pushed against the top of the cassette device 100. This allows the efficient heating of the fluid in the fluid conduit to 37° C. There are holes in the heating element to allow light to pass. Pumps will be used to periodically move the fluid backwards and forwards along the channel by one or two millimetres to ensure all of the fluid in the channel is heated uniformly.

In yet another embodiment, all inlets are on the top of the cassette. All inlets are formed by piercing a membrane adhered to the either the top or bottomeither the top or bottom of the cassette with an injection needle. The membrane may be a sheet of silicone rubber.

In still another embodiment of the present invention, the reservoirs contain wash and the reagents required to trigger the chemiluminescence reaction. The reagents are stored in bottles with septum lids. The bottles are combined and formed into a complete reagent package which is inserted into the instrument. On insertion injection needles pierce all of the septum membranes.

Small bore (0.5 to 1.5 mm inside diameter) silicone, neoprene or bioprene tubing carries the various liquids to their corresponding pumps and forward to the corresponding inlet port of the cassette. The pumps are required to pump up to 200 μL from the reagent bottles to the cassette. The pumps should be either syringe or peristaltic because these are capable of delivering a specific volume. The volume delivered in a particular assay process will be monitored by determining how far the pump mechanism has moved. This is done using optical reflective switches to determine how far the peristaltic pump rotates or how far the plunger has been depressed in a syringe pump. In both cases a geared dc motor is used mainly for simplicity and compactness, alternatively a stepping motor may be used. Because a syringe pump is capable of giving a more accurate and precise dose (the diameter of the peristaltic tubing may change slightly over time) this may be preferred where small precise doses are required to activate one bead at a time.

In yet another embodiment of the present invention, the optical sensor is either a photomultiplier tube or a “multi pixel photon counting detector”, or a “silicone photo multiplier”.

In another embodiment the sensor will be of the silicon photomultiplier type. In order to achieve adequate signal to noise with these devices they must be cooled to reduce the dark current and the temperature must be monitored so that the average dark signal might be subtracted from the actual signal. The proposed system is shown in FIG. 4. A fan 401, a heat sink 402 and a peltier 403 are used to cool the sensor board 404, which also contains a temperature sensor. Enclosure 405 and glass window 406 are included to ensure that the sensor electronics are not subject to condensation.

In an embodiment of the present invention, the photo-detector (either a photon counting photomultiplier tube or a cooled “silicon PMT”) is placed directly above each well sequentially, geared direct current (DC) motors are used to rotate the light sensor into position. In case when the fluid conduit forms a circular path on the cassette, the motor is moved in a circular path around a fulcrum.

In another embodiment in the present invention, the beads may be placed in a grid pattern and the photomultiplier would be moved in a rectilinear fashion using geared DC motors, lead screws and linear bearings. In both cases mechanical switches or reflective optical sensors are be used to determine the position.

In yet another embodiment, the heating element is an infrared LED emitting at 1500 nm or a radiative infrared emitter. At this wavelength water is quite absorbent and so it may be possible to heat the very small volume (<10 μL) sufficiently using a non-contact 304 method. A non-contact thermometer would be used to monitor the temperature.

In still another embodiment, a heating element 205 along with the injection needles 208 move as a unit to engage with the cassette device 100.

In yet another preferred embodiment of the present invention, a method of automated quantitative assay is disclosed having following steps: obtaining a cassette fully loaded with Beads and antibody/conjugate cocktail,—injecting a sample into a port at or upstream of the mixing chamber and filling the mixing chamber, the sample may be a number of body fluids including blood, saliva, and urine, inserting the cassette into the measuring instrument, activating the magnetic stirrer to mix the sample and the conjugate/antibody cocktail, pumping a neutral liquid or air into the mixing chamber causing the sample/conjugate/antibody mixture to be displaced and to flow round the fluid conduit immersing the beads, heating the liquid to approximately 37° C. to allow incubation, pumping the wash through fluid conduit to remove all of the remaining sample not bound to the beads, repeating the wash cycles and purging the fluid conduit of liquid by pumping air through the conduit between the wash cycles, measuring the chemiluminescence through sensors and recording the result, ejecting the cassette from the measuring instrument and shutting down the device.

There are then 2 alternative assay types that can be utilized in the proposed system—

1. If the chemiluminescence is of a ‘glow’ type. The activating reagents are pumped round the whole fluid conduit and then the light detector is moved to detect light from each of the beads in turn.

2. If the chemiluminescence is of a ‘flash’ type. It must be pumped so that it immerses each bead in turn and the luminescence must be detected before it is pumped to immerse the next bead.

In yet another embodiment of the present invention, the magnetic stirrer is activated by placing a motor comprising a magnet attached to shaft below the mixing chamber and wherein the magnet and a shaft are rotated by the motor thereby resulting in the rotation of the small magnet in the mixing chamber.

In still another embodiment of the present invention said device and the assay is used for the detection of novel coronavirus 2019-nCoV, SARS-CoV2 or viral or bacterial outbreak, antigens; antibodies, particularly those induced in response to an infection, allergic reaction, or vaccine; hormones, proteins and other physiological substances for example, human chorionic gonadotropin, estrogens, progestins, testosterones, corticosteroids, human growth factors, hemoglobin, and cholesterol; nucleic acids; a variety of enzymes; therapeutic compounds and illicit drugs; contaminants and environmental pollutants; or any number of natural or synthetic substances; ACE inhibitors, adrenergics and anti-adrenergics, alcohol deterrents for example, disulfiram, anti-allergics, anti-anginals, anti-arthritics, anti-infectives including antibacterials, antibiotics, antifungals, antihelminthics, antimalarials and antiviral agents, analgesics and analgesic combinations, local and systemic anesthetics, appetite suppressants, antioxidants, anxiolytics, anorexics, antiarthritics, anti-asthmatic agents, anticoagulants, anticonvulsants, antidiabetic agents, antidiarrheals, anti-emetics, anti-epileptics, antihistamines, anti-inflammatory agents, antihypertensives, antimigraines, antinauseants, antineoplastics, antioxidants, antiparkinsonism drugs, antipruritics, antipyretics, antirheumatics, antispasmodics, antitussives, adrenergic receptor agonists and antagonists, anorexics, appetite suppressants, cardiovascular preparations including anti-arrhythmic agents, cardiotonics, cardiac depressants, calcium channel blockers and beta blockers), cholinergics and anticholinergics, contraceptives, diuretics, decongestants, growth stimulants, herbal preparations, hypnotics, immunizing agents, immunomodulators, immunosuppressives, muscle relaxants, neurologically-active agents including anti-anxiety preparations, antidepressants, antipsycotics, psychostimulants, sedatives and tranquilizers, sore throat medicaments, sympathomimetics, vasodilators, vasoconstrictors, vitamins, xanthine derivatives, various combinations of these compounds, and the like.

The following examples further illustrate the invention and its unique characteristics in elaborate manner. However, the example in no way intend to limit the scope of the invention.

### EXAMPLE 1

### TSH Assay

### Method Summary

A Thyroid stimulating hormone (TSH) assay was developed on the Quantilyte system using Quantilyte beads but washed and read in 96-well plate as a standard ELISA format. The method for each assay was kept as similar as possible. Same conjugate, calibrator and QC preparations were used for each method. Briefly, Quantilyte beads were coated with 20 μg/ml of Goat-anti-TSH Nunc F (US Biologicals). ELISA wells were coated with 5 μg/ml of the same antibody. TSH was spiked into TSH depleted plasma to create a calibration line covering a range of 2905-1.90 IU/ml and quality control samples at 7.5, 15, 50, 300 and 1800 IU/ml. These were aliquoted and stored frozen at −20° C. Samples were analysed by mixing 25 μl of serum sample with 25 μl of conjugate comprising 0.5 μg/ml USB mouse-anti-TSH and 1 μg/ml anti-mouse-HRP. This mixture was incubated with coated beads or wells for 20 minutes before washing. Thereafter pierce supersignal pico or TMB substrate was added and reading was taken using the POLARSTAR plate reader of the Quantilyte reader. Calibrators were analysed in triplicate and average of calibration lines was used to calculate concentration for six replicate quality control samples. % CV and % Bias was calculated for each QC level. Method sensitivity was determined by running six replicate zero calibrators determined mean+three standard deviations and finding a concentration for this value by reading from the averaged calibration lines.

### Calibration Lines Comparison

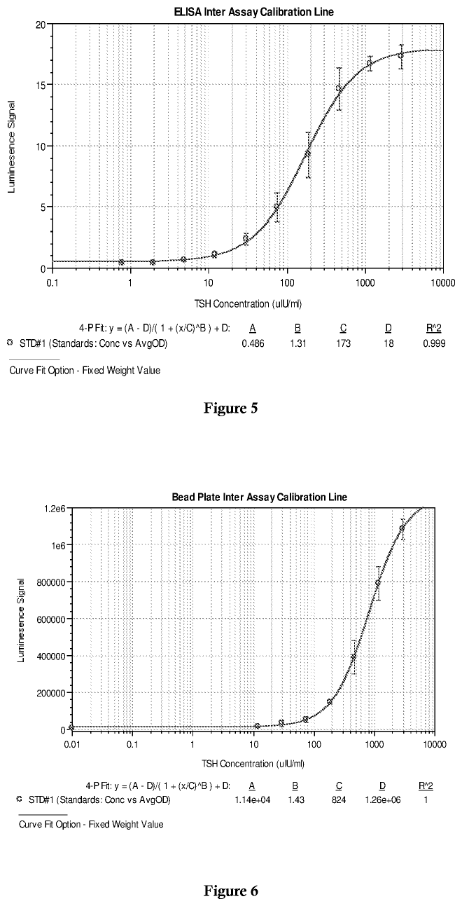

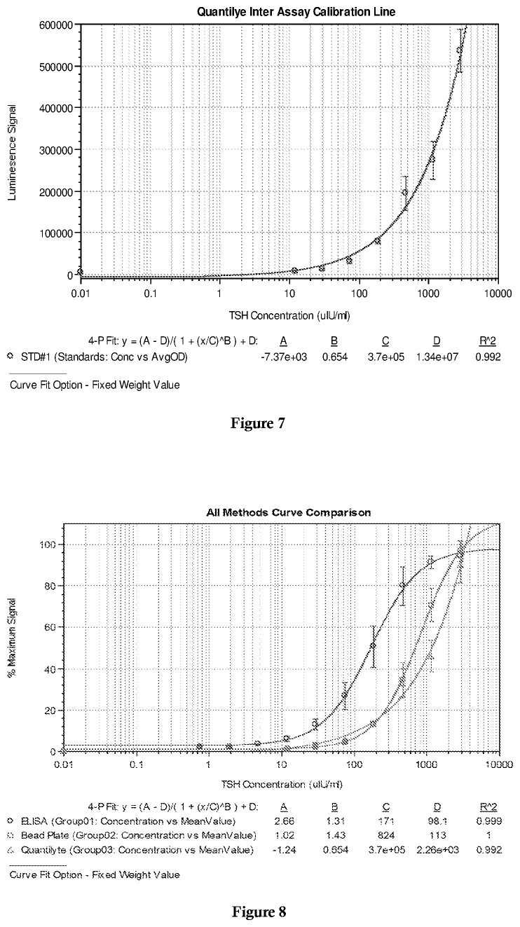

Table 1 shows the Mean Signal, %CV and % Maximum Value for the triplicate calibration lines. Graphs shown in FIGS. 5, 6, 7 and 8 below show each line plotted individually as mean signal with standard deviation error bars and combined shown % of maximum value. It can be seen that the Quantilyte beads produce a very similar shape of curve when analysed using the POLARSTAR reader or the Quantilyte reader. Absolute values are approximately 50% lower on the Quantilye reader. The use of a black cartridge compared to a white plate and the presence of a film surface over the cartridge may account for this difference. The ELISA line shows significantly greater separation of signal values at lower TSH levels and hence greater sensitivity this is likely to be accounted for by the high binding surface of the ELISA plate compared to the standard polystyrene beads a high binding surface will hold more protein per mm of surface area and thus bind more analyte.

TABLE 1
Mean Signal, % CV and % Maximum Value for the triplicate calibration lines of TSH assay
All Methods Mean Calibration Lines

| | | ELISA | | | Bead Plate | | | Quantilyte | | |
|---|---|---|---|---|---|---|---|---|---|---|
| | | Mean | | % Max | Mean | | % Max | Mean | | % Max |
| uIU/ml | ng/ml | Signal | % CV | Signal | Signal | % CV | Signal | Signal | % CV | Signal |
| 0.01 | 0 | 0.375 | 21.9 | 2.17 | 1265 | 22.1 | 0.117 | 3323 | 67.8 | 0.620 |
| 1.90 | 0.224 | 0.393 | 13.8 | 2.27 | NA | NA | NA | NA | NA | NA |
| 4.76 | 0.560 | 0.605 | 8.25 | 3.50 | NA | NA | NA | NA | NA | NA |
| 11.9 | 1.40 | 1.12 | 21.1 | 6.50 | 12155 | 37.3 | 1.12 | 6783 | 40.9 | 1.27 |
| 29.7 | 3.50 | 2.37 | 19.2 | 13.7 | 30745 | 53.4 | 2.83 | 11947 | 17.2 | 2.23 |
| 74.4 | 8.76 | 4.94 | 24.0 | 28.5 | 51367 | 27.0 | 4.74 | 30816 | 18.6 | 5.75 |
| 186 | 21.9 | 9.26 | 19.9 | 53.6 | 148653 | 7.29 | 13.7 | 79636 | 7.52 | 14.9 |
| 465 | 54.7 | 14.6 | 11.7 | 84.7 | 388563 | 23.2 | 35.8 | 194890 | 20.9 | 36.4 |
| 1162 | 137 | 16.7 | 3.41 | 96.7 | 792668 | 11.4 | 73.1 | 273378 | 16.3 | 51.0 |
| 2905 | 342 | 17.3 | 5.70 | 100 | 1084667 | 5.10 | 100 | 535624 | 9.67 | 100 |

| | | | |
|---|---|---|---|
| S/N | 46.2 | 857 | 161 |
| r2 | 0.999 | 1.000 | 0.992 |

### Quality Control (QC) Sample Comparison

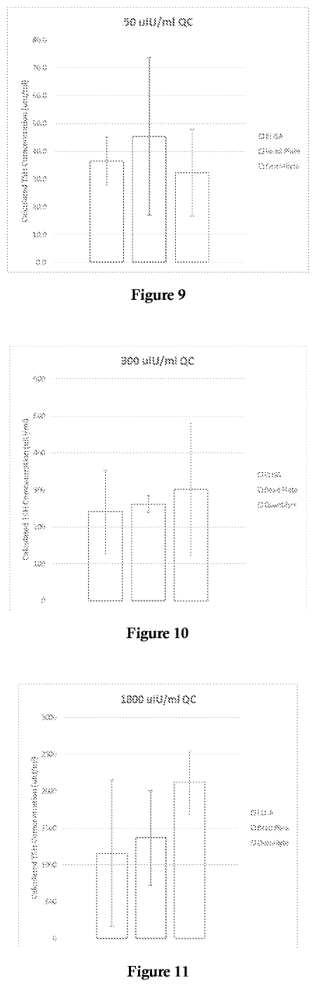

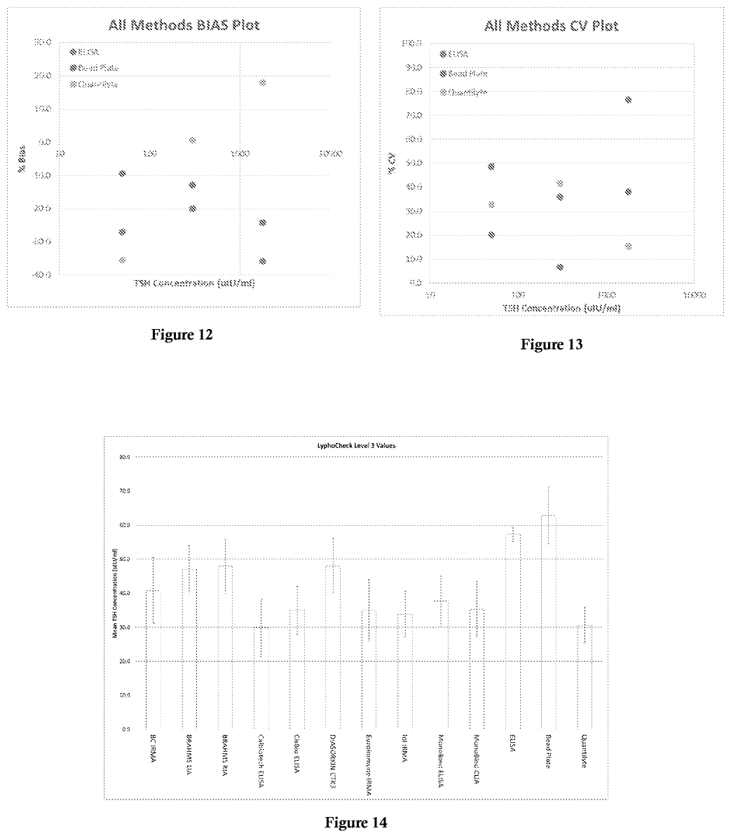

Table 2 shows the Mean, % CV range and % Bias of calculated TSH concentrations for quality control samples spiked at 50, 300 and 1800 μIU/ml analysed using all three methods additional quality control samples at 7.5 and 15 μIU/ml were analysed in the ELISA only due to the increased sensitivity of this method. The results for each quality control level found using all three methods are shown in FIGS. 9-11. It can be seen that the mean result and range are broadly comparable for all three methods. The Quantilyte method provides results for the quality control samples which are in line with those found using the ELISA method. The % BIAS and % CV values for each method at each of the three main QC levels are shown in graphs of FIGS. 12 and 13. It can be seen from these graphs that the Quantilyte assay does not show significantly higher variation or greater bias than the ELISA assay. However, the Quaniltyte assay does show a bias pattern showing negative bias (reading low) for low concentrations and positive bias (reading high) for higher concentrations. Similar pattern was observed in the TT4 data, suggesting this may represent a structural issue with the Quantilyte system.

TABLE 2
Mean, % CV range and % Bias of calculated TSH concentrations for quality control samples of TSH assay
All Methods Spiked QC's Mean Data

| | | ELISA | | | | Bead Plate | | | | Quantilyte | | | |
|---|---|---|---|---|---|---|---|---|---|---|---|---|---|
| uIU/ml | ng/ml | Mean | % CV | Range | % Bias | Mean | % CV | Range | % Bias | Mean | % CV | Range | % Bias |
| 7.5 | 0.883 | 5.58 | 84.1 | 5.64 | −25.5 | NA | NA | NA | NA | NA | NA | NA | NA |
| 15 | 1.77 | 9.66 | 31.1 | 3.56 | −35.6 | NA | NA | NA | NA | NA | NA | NA | NA |
| 50 | 5.89 | 36.5 | 20.0 | 8.65 | −27.1 | 45.3 | 48.7 | 28.4 | −9.34 | 32.3 | 32.8 | 15.7 | −35.5 |
| 300 | 35.3 | 240 | 35.8 | 112.9 | −20.0 | 261 | 6.64 | 22.5 | −12.9 | 302 | 41.7 | 178 | 0.630 |
| 1800 | 212 | 1154 | 76.5 | 998 | −35.9 | 1364 | 38.1 | 645 | −24.2 | 2122 | 15.2 | 427 | 17.9 |

### Lyphocheck Sample Comparison

Table 3 shows the mean, % CV range and % Bias calculated for the lyphocheck control samples. Level 3 was analysed using all three methods and levels 1 and 2 were analysed by ELISA only due to increased sensitivity of this method. The results for lyphocheck level 3 found using all three methods are shown in FIG. 14. It can be seen that the mean result and % CV are broadly comparable for all three methods the Quantilyte method provides results for the quality control samples which are in line with those found using the ELISA method. The ELISA method produces a result for lyphocheck level 2 though with significantly increased % Bias it does not produce a result for lyphocheck level 1. A comparison of the mean and range calculated for lyphocheck level 3 using the ELISA, Bead Plate and Quantilyte methods and a variety of commercially available TSH assays (taken from the product literature) is shown on the next graph it can be seen that the Quantilyte system produces a result which is broadly in line with other commercially available assays both the bead plate and ELISA assays produce results which are slightly elevated relative to the range of commercial assays.

TABLE 3
Mean, % CV range and % Bias calculated for
the lyphocheck control samples of TSH assay
LyphoCheck Level 3 Data

| | BC | BRAHMS | BRAHMS | Calbiotech | CisBio | DIASORKIN |
|---|---|---|---|---|---|---|
| Test | IRMA | LIA | RIA | ELISA | ELISA | CTK3 |
| Mean | 40.8 | 47.0 | 48.0 | 29.9 | 35.0 | 48.0 |
| Range | 9.7 | 7.0 | 8.0 | 8.4 | 7.0 | 8.0 |

| | EuroImmune | lol | MonoBind | MonoBind | | Bead | |
|---|---|---|---|---|---|---|---|
| Test | IRMA | IRMA | ELISA | CLIA | ELISA | Plate | Quantilyte |
| Mean | 35.0 | 33.8 | 37.7 | 35.4 | 57.3 | 62.9 | 30.6 |
| Range | 9.0 | 6.8 | 7.4 | 8.2 | 2.2 | 8.4 | 5.3 |

### Minimum Detectable Concentration Comparison

Table 4 below shows the data and calculation performed to determine minimum detectable TSH concentration for each of the assay methods. Six zero samples were run in each method. Mean and standard deviation of these were found and a mean+3 times standard deviation signal was calculated and read for the calibration lines for each method to give the minimum detectable concentration. It can be seen that the minimum detectable concentration is similar using the ELISA assay and the Bead plate assay. However, the Quantilyte assay has an increased detection limit. This is likely to be due to slightly increased variation from the replicate zero samples in the Quantilyte assay which could reflect a less thorough and consistent wash procedure than the other methods.

TABLE 4
Data and calculation performed to determine minimum detectable
TSH concentration for each of the assay methods.

| | ELISA | Bead Plate | Quantilyte |
|---|---|---|---|
| Rep 1 | 0.439 | 1463 | 1587 |
| Rep 2 | 0.464 | 1343 | 4912 |
| Rep 3 | 0.479 | 1121 | 2595 |
| Rep 4 | 0.381 | 2530 | 1897 |
| Rep 5 | 0.469 | 2633 | 2965 |
| Rep 6 | 0.528 | 1652 | 4874 |
| Mean | 0.460 | 1790 | 3138 |
| StDev | 0.0485 | 637 | 1444 |
| 3*Stdev | 0.146 | 1912 | 4333 |
| Mean + 3*stdev | 0.606 | 3703 | 7471 |
| Minimum | 3.87 | 4.16 | 11.3 |
| Detectable | | | |
| Concentation | | | |
| (uIU/ml) | | | |
| Minimum | 0.455 | 0.490 | 1.33 |
| Detectable | | | |
| Concentation | | | |
| (ng/ml) | | | |

### EXAMPLE 2

### TT4 & Cortisol Multiplex Assay

### Method Summary

A Multiplex assay was developed on the Quantilyte system to detect TT4 and Cortisol in a single cartridge. Results obtained from this assay were compared to separate commercial ELISA assays for TT4 and cortisol obtained from Alpco. Briefly, the Quantilyte assay consisted of Quantilyte beads which were coated at 20 μg/ml with either anti-T4 or anti-Cortisol antibody. T4 and cortisol were spiked into T4 or cortisol depleted serum respectively to create independent TT4 and cortisol calibration lines which were aliquoted and stored frozen at −20° C. Calibrators, Lyphocheck QC's and cat serum samples were analysed by adding 90 μl of serum sample and 90 μl of assay buffer to 20 μl of conjugate concentrate containing 60 μg/ml T4-HRP and 1/10 dilution of cortisol-HRP in an HRP stabilising buffer. This mixture was incubated with coated beads for 20 mins before washing and adding Pierce supersignal pico substrate and reading using the Quantilyte reader. The Alpco ELISAs were conducted according to the kit instructions which being a colorimetric TMB ELISA was read using the POLARSTAR plate reader. Calibrators were analysed in triplicate and averaged calibration lines were used to calculate concentration for four replicates. % CV and % Bias of Lyphocheck quality control samples and 41 cat serum samples was calculated for each QC level.

### Calibration Lines Comparison

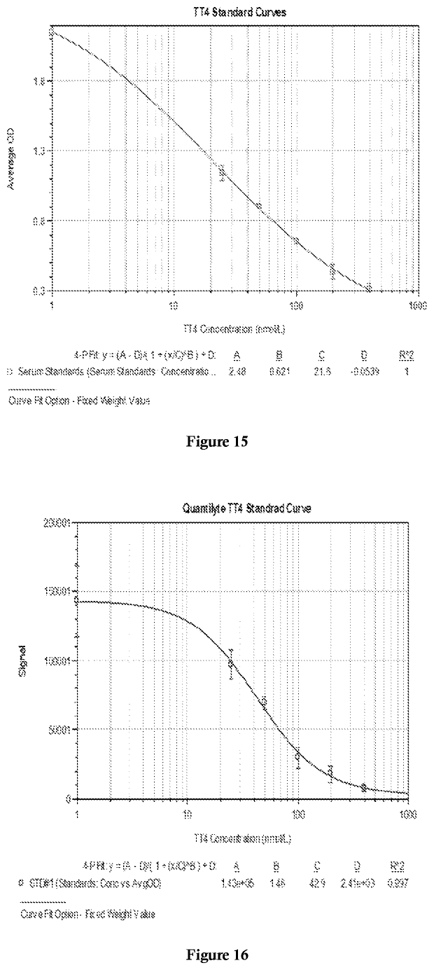

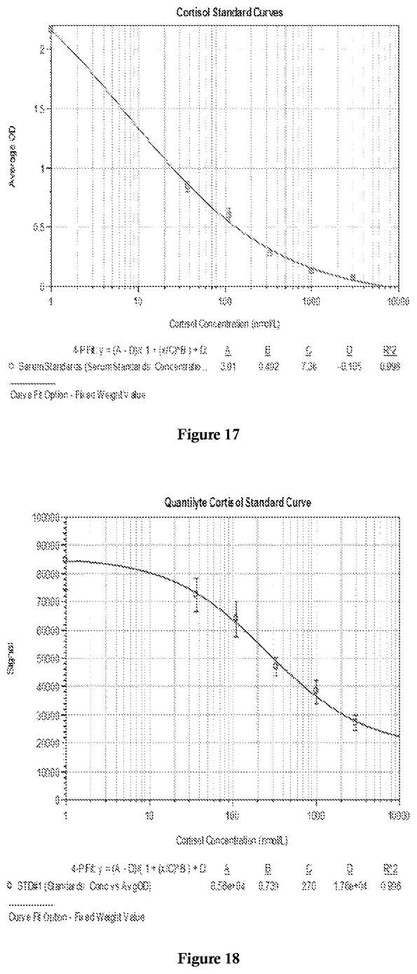

Table 5 and Table 6 below show the Mean Signal, % CV and % Maximum Signal for the triplicate TT4 and cortisol calibration lines respectively. FIGS. 15, 16, 17 and 18 show each line plotted individually as mean signal with standard deviation error bars. It can be seen that the Quantilyte beads produce rather different shape of curve to the ELISA for both TT4 and cortisol. The Quantilyte line show greater signal to noise in the TT4 assay however this pattern is reversed in the cortisol assay. The reason for this would seem to be that the multiplex format compromises the cortisol assay more than the TT4 with a greater amount of cortisol-HRP being required to produce a useable signal and this has compromised sensitivity.

TABLE 5
Mean Signal, % CV and % Maximum Signal for the triplicate TT4 calibration lines
TT4 Quantilye vs ELISA Calibration Lines

| | | ELISA | | | | Quantilyte | | | |
|---|---|---|---|---|---|---|---|---|---|
| Nominal T4 | Nominal T4 | | | | % | | | | % |
| Concentation | Concentation | Mean | Standard | | Maximum | Mean | Standard | | Maximum |
| (nmol/L) | (ng/ml) | Signal | Deviation | % CV | Signal | Signal | Deviation | % CV | Signal |
| 1 | 0 | 2.15 | 0.0225 | 1.05 | 100 | 142983 | 25815 | 18.1 | 100 |
| 25.0 | 19.4 | 1.15 | 0.0541 | 4.72 | 53.3 | 97088 | 10719 | 11.0 | 67.9 |
| 50.0 | 38.8 | 0.905 | 0.0156 | 1.72 | 42.0 | 69318 | 4908 | 7.08 | 48.5 |
| 100 | 77.5 | 0.654 | 0.0178 | 2.72 | 30.4 | 29599 | 7776 | 26.3 | 20.7 |
| 200 | 155 | 0.438 | 0.0528 | 12.1 | 20.3 | 17801 | 5602 | 31.5 | 12.4 |
| 400 | 310 | 0.311 | 0.0422 | 13.6 | 14.4 | 7562 | 2212 | 29.3 | 5.29 |

| | | |
|---|---|---|
| Signal to Noise | 6.92 | 18.9 |

TABLE 6
Mean Signal, % CV and % Maximum Signal for the triplicate cortisol calibration lines
Cortisol Quantilye vs ELISA Calibration Lines

| Nominal | Nominal | ELISA | | | | Quantilyte | | | |
|---|---|---|---|---|---|---|---|---|---|
| Cortisol | Cortisol | | | | % | | | | % |
| Concentation | Concentation | Mean | Standard | | Maximum | Mean | Standard | | Maximum |
| (nmol/L) | (ng/ml) | Signal | Deviation | % CV | Signal | Signal | Deviation | % CV | Signal |
| 1 | 0 | 2.16 | 0.149 | 6.86 | 100 | 84520 | 10137 | 12.0 | 100 |
| 33.3 | 12.1 | 0.841 | 0.0431 | 5.12 | 38.9 | 72447 | 5850 | 8.08 | 85.7 |
| 111 | 40.2 | 0.601 | 0.0587 | 9.77 | 27.8 | 63872 | 6242 | 9.77 | 75.6 |
| 333 | 121 | 6.273 | 0.0130 | 4.76 | 12.6 | 47026 | 3407 | 7.24 | 55.6 |
| 1000 | 362 | 0.130 | 0.0142 | 10.9 | 6.01 | 38075 | 4068 | 10.7 | 45.0 |
| 3000 | 1086 | 0.0697 | 0.0107 | 15.3 | 3.22 | 27020 | 2725 | 10.1 | 32.0 |

| | | |
|---|---|---|
| Signal to Noise | 31.1 | 3.13 |

### Lyphocheck QC Sample Comparison

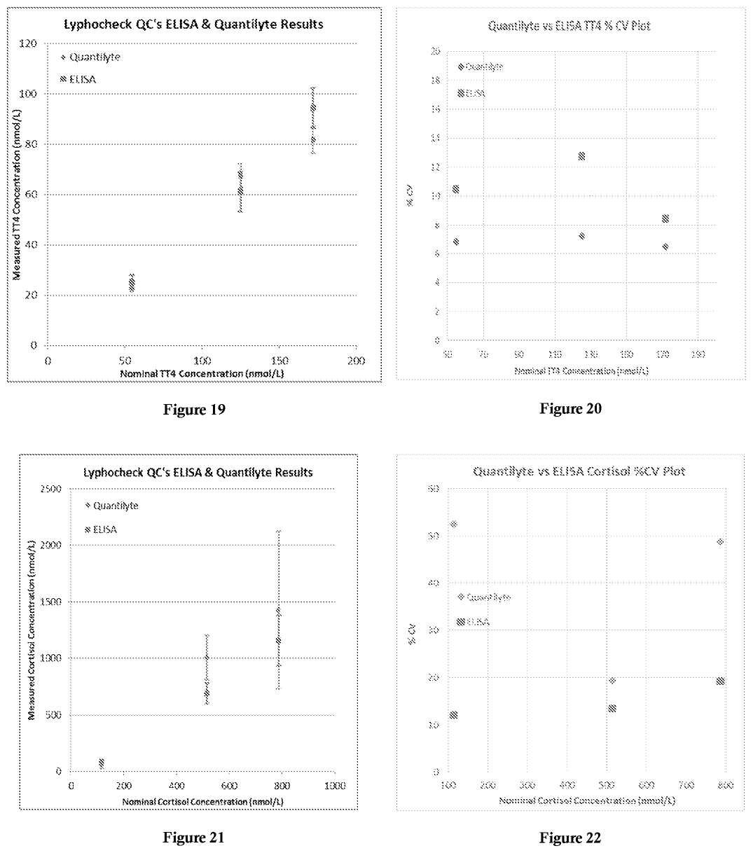

Table 7 and 8 below shows the mean calculated concentration, % CV range and % Bias calculated for the Lyphocheck control samples for TT4 and cortisol samples respectively as well as the % difference between the concentrations calculated using the Quantilyte and ELISA methods. The mean concentration with standard deviation error bars at each QC level for both methods are plotted on the graphs shown in FIGS. 19 and 21 along with the % CV at each level for both methods in the graph of FIGS. 20 and 22. For the TT4 assay it can be seen that the mean result and % CV are broadly comparable for both methods at all three QC levels with the Quantilyte assay showing marginally lower % CV values than the ELISA. For the cortisol assay although the mean results are comparable although more widely spread than for TT4 the Quantilyte assay show significantly higher % CV than the ELISA assay this is likely to be due to the much reduced signal to noise of the Quantilyte assay which exaggerates small signal variation into large differences in the calculated concentration.

TABLE 7
Mean calculated concentration, % CV range and % Bias calculated for the Lyphocheck control samples for TT4 samples
TT4 Quantilye vs ELISA LYPHOCHECK QC Samples

| | | ELISA | | | | Quantilyte | | | | |
|---|---|---|---|---|---|---|---|---|---|---|
| | | Mean | | | | Mean | | | | % |
| Nominal T4 | Nominal T4 | Calculated | | | | Calculated | | | | Difference |
| Concentation | Concentation | Concentration | Standard | | | Concentration | Standard | | | Quantilye |
| (nmol/L) | (ng/ml) | (nmol/L) | Deviation | % CV | % Bias | (nmol/L) | Deviation | % CV | % Bias | vs ELISA |
| 54.6 | 42.3 | 25.4 | 2.66 | 10.5 | −53.6 | 23.0 | 1.57 | 6.8 | −57.9 | −4.90 |
| 125 | 96.9 | 61.2 | 7.81 | 12.8 | −51.0 | 67.6 | 4.88 | 7.2 | −45.9 | 4.96 |
| 172 | 133 | 94.5 | 7.96 | 8.43 | −45.1 | 81.8 | 5.29 | 6.47 | −52.4 | −7.17 |

TABLE 8
Mean calculated concentration, % CV range and % Bias calculated for the Lyphocheck control samples for cortisol samples
Cortisol Quantilye vs ELISA LYPHOCHECK QC Samples

| | | ELISA | | | | Quantilyte | | | | |
|---|---|---|---|---|---|---|---|---|---|---|
| Nominal | Nominal | Mean | | | | Mean | | | | % |
| Cortisol | Cortisol | Calculated | | | | Calculated | | | | Difference |
| Concentation | Concentation | Concentration | Standard | | | Concentration | Standard | | | Quantilye |
| (nmol/L) | (ng/ml) | (nmol/L) | Deviation | % CV | % Bias | (nmol/L) | Deviation | % CV | % Bias | vs ELISA |
| 115.0 | 41.6 | 87.2 | 10.5 | 12.1 | −24.2 | 57.9 | 30.4 | 52.5 | −49.7 | −20.2 |
| 514 | 186.1 | 691 | 92.9 | 13.4 | 34.4 | 1007 | 196 | 19.4 | 95.8 | 18.6 |
| 787 | 285 | 1159 | 223 | 19.2 | 47.3 | 1424 | 697 | 48.9 | 80.9 | 10.2 |

### Cat Serum Sample Comparison

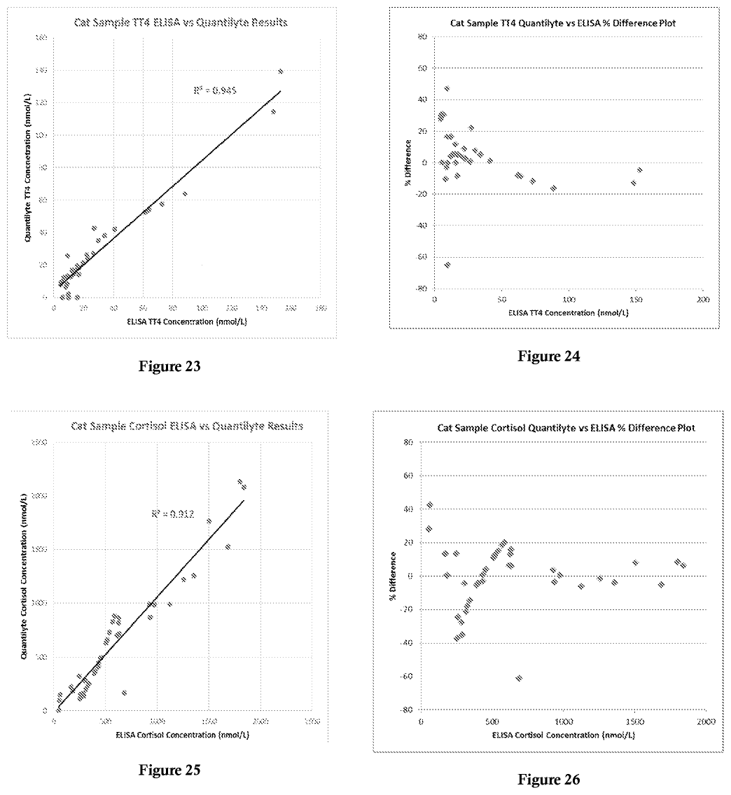

Table 9 below shows the calculated TT4 and cortisol concentrations for 41 cat serum samples found using the both the Quantilyte and ELISA methods as well as the % difference between the result found using the Quantilyte assay and that using the ELISA assay. The graphs shown in FIGS. 23 and 25 below show plots of the ELISA results vs the Quantilyte results and FIGS. 24 and 26 show a scatter graph of the percentage differences vs the concentration determined by ELISA for TT4 and cortisol respectively.

The data shows reasonable correlation of results from Quantilyte and ELISA assay for both the TT4 and Cortisol methods with correlations of 0.945 and 0.912 respectively. While there is considerable variation among individual results along with a few extreme outliers the two methods are broadly in agreement with respect to the relative concentrations of the samples for both analytes. Both analytes show a tendency to increased variation at low analyte concentrations and the TT4 assay shows a pronounced tendency to produce higher value for sample below 20 nmol/L than the ELISA.

TABLE 9
Calculated TT4 and cortisol concentrations for 41 cat serum samples found using
the both the Ouantilvte and ELISA methods as well as the % difference between
the result found using the Ouantilvte assay and that using the ELISA assay
Cat Serum Sample TT4 and Cortisol Results

| | | | | | | | ELISA | Quantilyte | |
|---|---|---|---|---|---|---|---|---|---|
| | | | | ELISA | Quantilyte | | Calculated | Calculated | |
| | | | | Calculated TT4 | Calculated TT4 | % Difference | Cortisol | Cortisol | % Difference |
| | | | | Concentration | Concentration | Quantilyte | Concentred on | Concentratton | Quantilyte |
| Sample No | Prefix | Suffix | Letter | (nmol/L) | (nmol/L) | vs ELISA | (nmol/L) | (nmol/L) | vs ELISA |
| 1 | 1002907 | 66 | S | 8.98 | 8.45 | −3.03 | 183 | 185 | 0.369 |
| 2 | 1002837 | 113 | S | 22.1 | 26.4 | 8.80 | 539 | 729 | 15.0 |
| 3 | 102907 | 154 | S | 9.75 | 2.08 | −64.8 | 245 | 321 | 13.5 |
| 4 | 1002863 | 214 | S | 62.0 | 52.8 | −8.02 | 507 | 634 | 11.1 |
| 5 | 1002837 | 261 | U | 11.8 | 12.8 | 4.18 | 1685 | 1526 | −4.96 |
| 6 | 1002837 | 268 | S | 10.0 | Range? | NC | 285 | 162 | −27.7 |
| 7 | 1002837 | 268 | S | 15.5 | 19.6 | 11.8 | 168 | 221 | 13.5 |
| 8 | 1002837 | 269 | S | 27.3 | 42.8 | 22.0 | 628 | 820 | 13.2 |
| 9 | 1002837 | 269 | S | 30.0 | 35.1 | 7.83 | 975 | 988 | 0.677 |
| 10 | 1002837 | 270 | S | 34.3 | 38.0 | 5.11 | 517 | 664 | 12.4 |
| 11 | 1002837 | 270 | S | 41.1 | 42.1 | 1.23 | 306 | 281 | −4.28 |
| 12 | 1002867 | 326 | S | 15.8 | Range? | NC | 686 | 166 | −60.9 |
| 13 | 1002867 | 339 | S | 16.9 | 18.7 | 5.19 | 633 | 716 | 6.16 |
| 14 | 1002833 | 426 | S | 19.5 | 21.2 | 4.27 | 926 | 992 | 3.44 |
| 15 | 1002866 | 451 | 4 | 4.41 | 7.80 | 27.8 | 628 | 865 | 15.3 |
| 16 | 1002840 | 528 | S | 13.4 | 14.9 | 5.27 | 342 | 256 | −14.4 |
| 17 | 1002848 | 528 | U | 6.59 | 12.3 | 30.2 | 1357 | 1256 | −3.85 |
| 18 | 1002843 | 597 | S | 5.50 | Range? | NC | 253 | 115 | −37.3 |
| 19 | 1002835 | 631 | S | 12.2 | 17.0 | 16.5 | 61.5 | 153 | 42.6 |
| 20 | 1002835 | 631 | S | 5.03 | 9.43 | 30.4 | 48.8 | 4.37 | −83.6 |
| 21 | 1002814 | 638 | S | 73.0 | 57.6 | −11.8 | 1123 | 992 | −6.17 |
| 22 | 1002833 | 640 | S | 9.3 | 26.0 | 47.1 | 311 | 202 | −21.2 |
| 23 | 1002833 | 640 | S | 13.3 | 14.8 | 5.24 | 288 | 139 | −34.9 |
| 24 | 1002866 | 661 | 4 | 7.98 | 6.48 | −10.4 | 1800 | 2135 | 8.51 |
| 25 | 1002867 | 669 | S | 9.31 | 13.0 | 16.5 | 432 | 440 | 0.92 |
| 26 | 1002860 | 702 | S | 148 | 114 | −12.9 | 432 | 407 | −2.92 |
| 27 | 1002860 | 702 | S | 153 | 139 | −4.72 | 258 | 156 | −24.5 |
| 28 | 1002868 | 703 | S | 19.8 | 21.5 | 4.14 | 392 | 352 | −5.40 |
| 29 | 1002860 | 703 | S | 22.9 | 24.1 | 2.67 | 507 | 634 | 11.1 |
| 30 | 1002863 | 728 | S | 16.9 | 14.2 | −8.45 | 1501 | 1765 | 8.08 |
| 31 | 1002863 | 728 | 4 | 5.93 | Range? | NC | 325 | 226 | −18.0 |
| 32 | 1002832 | 802 | S | 13.2 | 14.7 | 5.21 | 454 | 493 | 4.13 |
| 33 | 1002832 | 802 | S | 16.9 | 18.8 | 5.18 | 399 | 367 | −4.22 |
| 34 | 1002832 | 802 | U | 5.21 | 9.85 | 30.8 | 934 | 869 | −3.59 |
| 35 | 1002867 | 812 | S | 26.6 | 27.0 | 0.744 | 615 | 699 | 6.40 |
| 36 | 1002865 | 835 | 4 | 88.6 | 63.9 | −16.3 | 1256 | 1223 | −1.34 |
| 37 | 1002835 | 840 | S | 14.8 | 16.6 | 5.50 | 517 | 664 | 12.4 |
| 38 | 1002835 | 840 | S | 13.7 | 15.2 | 5.35 | 585 | 884 | 20.3 |
| 39 | 1002816 | 891 | S | 64.1 | 53.7 | −8.80 | 569 | 828 | 18.5 |
| 40 | 1002811 | 911 | U | 13.7 | 15.3 | 5.36 | 1841 | 2083 | 6.17 |
| 41 | 1002841 | 978 | U | 4.89 | 9.09 | 30.0 | 54.5 | 97.4 | 28.2 |

### Anti-Cortisol Bead Coating Procedure

150 2 mm polystyrene beads were coated with 3 ml of 20 μg/ml of anti-Cortisol in 100 mM carbonate coating buffer pH 9.6 i.e. 7.5 μl of 7.91 mg/ml anti-Cortisol in dH₂O+2992.5 μl coat buffer. Added coated beads to a 5 ml Bijou bottle. Beads were incubated overnight at 4° C. with end-over-end mixing. Beads were washed eight times with 3 ml PBS/0.01% Tween and twice with 3 ml PBS and 3 ml of 50% StartingBlock in PBS+0.25M Trehalose Block buffer was added. Beads were incubated for two hours at room temperature with end-over-end mixing and then washed three times with 1 ml PBS/0.01% Tween and placed in a weigh boat and allowed to air dry at room temperature.

### Anti-T4 Bead Coating Procedure

150 2 mm polystyrene beads are coated with 3 ml of 20 μg/ml of anti-Cortisol in 100 mM carbonate coating buffer pH 9.6 i.e. 31.4 μl of 1.91 mg/ml anti-Cortisol in dH₂O+2968.6 μl coat buffer. Added coated beads to a 5 ml Bijou bottle. Beads were incubated overnight at 4° C. with end-over-end mixing. Beads were washed eight times with 3 ml PBS/0.01% Tween and twice with 3 ml PBS and 3 ml of 50% StartingBlock in PBS+0.25M Trehalose Block buffer was added. Beads were then incubated for two hours at room temperature with end-over-end mixing and then washed three times with 1 ml PBS/0.01% Tween and placed in a weigh boat and allowed to air dry at room temperature.

### Buffer Solution Preparation

Buffer solution is prepared a blocking reagent and the binding release components, 8-Anilinonaphthalene-1-sulfonic acid (ANS) and sodium salicylate.

400 μl of 50 mg/ml ANS+100 μl of 200 mg/ml NaS+9500 μl of AbCam IM Block Final Concentrations=2 mg/ml ANS, 2 mg/ml Sodium Salicylate

### Conjugate Mix Preparation

Conjugate solution is prepared containing Cortisol-HRP and anti-T4-HRP in an HRP stabilizing buffer

100 μl of cortisol-HRP+10 μl of 6000 nmol/L anti-T4-HRP+890 μl of KPL HRP stabilizer

Final Concentrations= 1/10 Cortisol-HRP+60 nmol/L T4-HRP

### TT4 Serum Calibrators Preparation

T4 is serially diluted into T4 depleted serum as below in Table 10.

TABLE 10
TT4 Serum calibrators preparation

| | | | | Spiking | |
|---|---|---|---|---|---|
| T4 | T4 | | | Solution | Serum |
| Concentration | Concentration | Spiking | | Volume | Volume |
| (nmol/L) | (ng/ml) | Solution | | (μl) | (μl) |
| 400 | 310 | 129 | nmol/ml | 3.7 | 1196.3 |
| | | | Stock | | |
| 200 | 155 | 400 | nmol/L | 600 | 600 |
| 100 | 75.5 | 200 | nmol/L | 600 | 600 |
| 50 | 38.8 | 100 | nmol/L | 600 | 600 |
| 25 | 19.4 | 50 | nmol/L | 600 | 600 |

| | | | | |
|---|---|---|---|---|
| 0 | 310 | NA | NA | 1200 |

Calibrators stored at 4° C. for 24 hours then aliquoted and stored at −20° C.

### Cortisol Serum Calibrators Preparation

Cortisol is serially diluted into cortisol depleted serum as below.

TABLE 11
Cortisol serum calibration preparation

| | | | | Spiking | |
|---|---|---|---|---|---|
| Cortisol | Cortisol | | | Solution | Serum |
| Concentration | Concentration | Spiking | | Volume | Volume |
| (nmol/L) | (ng/ml) | Solution | | (μl) | (μl) |
| 100,000 Spike | 36231 Spike | 2,757,794 | | 7.3 | 192.7 |
| | | nmol/L Stock | | | |
| 3000 | 1086 | 100,000 | nmol/L | 36 | 1164 |
| 1000 | 362 | 3000 | nmol/L | 300 | 900 |
| 333 | 121 | 1000 | nmol/L | 300 | 900 |
| 111 | 40.2 | 333 | nmol/L | 300 | 900 |
| 33.3 | 13.4 | 111 | nmol/L | 300 | 900 |

| | | | | |
|---|---|---|---|---|
| 0 | 0 | NA | NA | 1200 |

Calibrators stored at 4° C. for 24 hours then aliquoted and stored at −20° C.

### Instrument Prime Method

Instrument wash bottle was filled with PBS/0.01% Tween+1/5000 antifoam wash solution and connected to instrument wash line.

An empty Prime cartridge was placed in the instrument.

The command WASH20 was run, followed by PURGE12, WASH12, PURGE12.

The prime cartridge was removed and emptied, the instrument was then ready for use.

### Assay Method

Anti-Cortisol bead was placed in well 1 and Anti-T4 bead was placed in well 2 of an eight well cartridge.

The cartridge was sealed using permanent heat seals using 2 ten second presses of the heat sealer.

A strip of self-adhesive silicon strip was secured over the cartridge injection port.

An air escape hole was pierced in the heat seal in the top right hand corner of the waste reservoir.

90 μl of serum calibrator was mixed with 90 μl of buffer solution and added to 20 μl of conjugate mix.

The serum sample/conjugate mix was injected through the first three wells of the cartridge.

The cartridge was incubated at room temperature for 20 minutes.

The cartridge was placed into the Quantiyte instrument and the wash sequence was run.

150 μl of Pierce supersignal pico substrate solution A was mixed with 150 μl of Pierce supersignal substrate solution B.

Cartridge was removed from the instrument 300 μl volume of mixed pierce supersignal pico substrate was injected into the cartridge.

Cartridge was incubated at room temperature for 2 minutes.

Cartridge was placed into the Quantilyte reader and luminescence signal recorded by running the read sequence.

### EXAMPLE 3

### C-Reactive Protein Assay

The assay consisted of beads which were coated at 5 μg/ml with anti-CRP capture antibody. CRP was spiked into CRP depleted serum to create independent CRP serum calibration lines and separately spiked serum quality control samples which were aliquoted and stored at 4° C. Serum calibrators and QCs were diluted 1 in 1000 in PBS and analysed by adding 180 μl of diluted serum sample to 20 μl of detection mix concentrate containing 900 ng/ml CRP detection antibody and 1/20 dilution of streptavidin-HRP in an HRP stabilising buffer. This mixture was incubated with coated beads for 10 mins before washing and adding Pierce supersignal pico substrate and reading using the instrument reader. The R&D systems ELISAs were conducted according to the kit instructions using the same antibodies used for the CRP assay and was a colorimetric TMB ELISA which was read using the POLARSTAR plate reader. A higher sample dilution of 1 in 125000 was required for the ELISA analysis. Calibrators were analysed in duplicate and averaged calibration lines were used to calculate concentration for six replicate quality control samples % CV and % Bias was calculated for each QC level.

### Methodology

2 mm Polystyrene beads were coated by passive adsorption with 5 μg/mg of CRP capture antibody, washed and stored in PBS. Beads were placed in individual wells of an eight well cassette. The cassette is sealed using permanent heat seals. Serum sample containing CRP was diluted and mixed 1:9 with conjugate solution containing CRP detection antibody labelled with streptavidin-HRP. Serum sample mix was injected through each used well of the cassette. Cassette was incubated at room temperature for 10 minutes. Cassette was placed into the measuring instrument and the Wash sequence was run. Pierce supersignal substrate was mixed and injected through each of the used wells and the cassette incubated for 2 minutes. Cassette is placed into the measuring instrument and the Read sequence is run. The luminescence signal from each of the used cassette wells is read in turn and recorded.

### Anti-CRP Bead Coating Procedure

One Hundred 2 mm polystyrene beads were coated with 3 ml of 5 μg/ml of anti-CRP in 100 mM carbonate coating buffer pH 9.6. Specifically, 41.6 μl of 360 μg/ml anti-CRP and 2958 μl coat buffer were used to coat the polystyrene beads. Added the coated 100 polystyrene beads in a 5 ml Bijou bottle and incubated overnight at 4° C. with gentle agitation. Beads were then washed four times with 3 ml PBS/0.01% Tween and twice with 3 ml PBS. Thereafter, the beads were stored in PBS buffer.

### Conjugate Mix Preparation

Conjugate solution was prepared containing Anti-CRP-Biotin detection antibody and streptavidin-HRP in a HRP-stabilising Buffer. Particularly, 55.5 μl of 16.2 μl g/ml Anti-CRP-Biotin, 50 μl of Streptavidin-HRP, 894.5 μl of HRP Stabilizer in a final concentration of 900 ng/ml Anti-CRP, 1 in 20 Streptavidin-HRP Conjugate Mix was stored at 4° C.

### CRP Serum Calibrators Preparation

CRP is serially diluted into CRP depleted serum as below in Table 11.

TABLE 11
CRP Serum Calibrators Preparation

| | | | Spiking | |
|---|---|---|---|---|
| CRP | | | Solution | Serum |
| Concentration | Spiking | | Volume | Volume |
| (μg/ml) | Solution | | (μl) | (μl) |
| 200 | 1 | mg/ml stock | 100 | 400 |
| 50 | 200 | μg/ml | 125 | 375 |
| 12.5 | 50 | μg/ml | 125 | 375 |
| 3.13 | 12.5 | μg/ml | 125 | 375 |
| 0.781 | 3.13 | μg/ml | 125 | 375 |

| | | | |
|---|---|---|---|
| 0.005 | NA | NA | 500 |

Calibrators are stored at 4° C.

### CRP Serum QC Preparation

CRP is serially diluted into CRP depleted serum as below in Table 12.

TABLE 12
CRP Serum QC preparation

| | | | Spiking | |
|---|---|---|---|---|
| CRP | | | Solution | Serum |
| Concentration | Spiking | | Volume | Volume |
| (μg/ml) | Solution | | (ul) | (ul) |
| 100 | 1 | mg/ml stock | 50 | 450 |
| 20 | 100 | μg/ml | 100 | 400 |
| 4 | 20 | μg/ml | 100 | 400 |

QC's are stored at 4° C.

### Calibrator/QC Dilution

Serum calibrators and QCs are diluted 1/1000in PBS prior to analysis as below:—

1/50-20 μl Calibrator/QC +980 μl PBS

1/1000-50 μl 1/50 Dilution+950 μl PBS

### Assay Method

Anti-CRP bead was placed in well 1 and well 2 of an eight well cassette along with 20 μl of PBS. The cassette was sealed using permanent heat seals using 2 ten second presses of the heat sealer. A strip of self-adhesive silicon strip was secured over the cassette injection port. An air escape hole was pierced in the heat seal in the top right hand corner of the waste reservoir. PBS was injected through all wells of the cassette as a storage solution. Air was injected to remove PBS storage solution from the wells. 180 ul of diluted calibrator or QC was mixed with 20 μl of conjugate solution. The serum sample/conjugate mix was injected through the first three wells of the cassette. The cassette was incubated at room temperature for 10 minutes. The cassette was placed into the measuring instrument and the wash sequence was run. 150 μl of Pierce super signal pico substrate solution A was mixed with 150 μl of Pierce super signal substrate solution B. Cassette was removed from the instrument 300 μl volume of mixed pierce super signal pico substrate was injected into the cassette. Cassette was incubated at room temperature for 2 minutes. Cassette is placed into the reader and luminescence signal was recorded by running the read sequence.

### Buffer/Stock Solution Preparation

### 10×PBS

Dissolve 80 g of NaCl, 2.0 g of KCl, 14.4 g of Na₂HPO₄ and 2.4 g of KH₂PO₄ in 1 L of dH₂O. Store at room temperature.

### PBS

Dilute 100 ml of 10×PBS with 900 ml of dH₂O

### PBS/0.01% Tween

Add 1 ml of 10% Tween to 990 ml of PBS

### PBS/0.01% Tween+ 1/5000 Anti-foam Wash Buffer

Add 20 μl of antifoam to 100 ml of PBS/0.01% Tween 10× Coating Buffer (1M Carbonate)

Dissolve 16.8 g of sodium hydrogen carbonate in 200 ml of dH₂O. Store at room temperature.

### 100 mM Carbonate Coating Buffer

Mix 50 ml of 10× x coat buffer with 450 ml of dH₂O

### Reagents and Equipment

### Antibody/Protein Reagents

| Reagent | Supplier | Cat No |
|---|---|---|
| Anti-CRP Capture Antibody | R&D Systems | 842676 |
| Anti-CRP Capture Antibody | R&D Systems | 842677 |
| Streptavidin-HRP | R&D Systems | 890803 |
| CRP Protein | USBiological | 214550 |

### Sera Reagents

| Reagent | Supplier | Cat No |
|---|---|---|
| Human CRP depleted serum | BBI | SF100-2 |

### Chemical Reagents

| Reagent | Supplier | Cat No |
|---|---|---|
| Antifoam S30 | Mistral | 150827 |
| NaCl | Sigma | 31434 |
| KCl | Sigma | P9333 |
| Na₂HPO₄ | Fisher | S42502/53 |
| KH₂PO₄ | Fisher | P4800/53 |
| Tween20 | Sigma | P1379 |
| Sodium hydrogen carbonate | Fisher | S/4240/53 |
| Supersignal ELISA Pico | Thermo | 37069 |
| 2 mm Polystyrene Beads | Cospheric | PSS 1.95 mm |
| Clear Seal Weld Mark II | 4titude | 4ti-0575 |

### Equipment

Scientific industries VotexGenie2
Gilson Pipetteman Pipettes (P1000, P200, P20)
Combi Thermosealer
Covilyte luminescence reader

### C-Reactive Protein Assay Analysis

### CRP Process

Preparation: Need supplies on hand:—syringe, heparin tube, sample pipette, conjugate pipette, high-speed centrifuge, untreated sample tube (from kit), conjugate (from kit). Device uses serum or plasma (optimal workflow=plasma)

### Sample Preparation

Dispense 1 cc of sample (freshly collected) into Lithium heparin tube

Invert sample 5 times

Place in centrifuge IMMEDIATELY

Spin for 2 mins (hard spin) or 10 mins (standard Lithium spin)

If using serum, blood must clot for a minimum of 20 mins before spinning

### Sample Transfer

Pipette 20 μl of plasma/serum sample into sample dilution tube containing 20 ml Buffer

Close tube and invert 5 times

Pipette 180 μl of diluted plasma/serum sample into sample tube

Add 20 μl of conjugate

Close tube and invert 5 times

### Incubation

Inject sample into cassette

Start timer

10 min incubation time

Timer alerts when incubation complete

### Run Test

Place cassette in device

Click “Run Full Procedure”

Device runs wash and read sequence CRP result is displayed in application

### Other Points to Note

Conjugate must be stored at 2-8° C.

### Calibration Lines Comparison

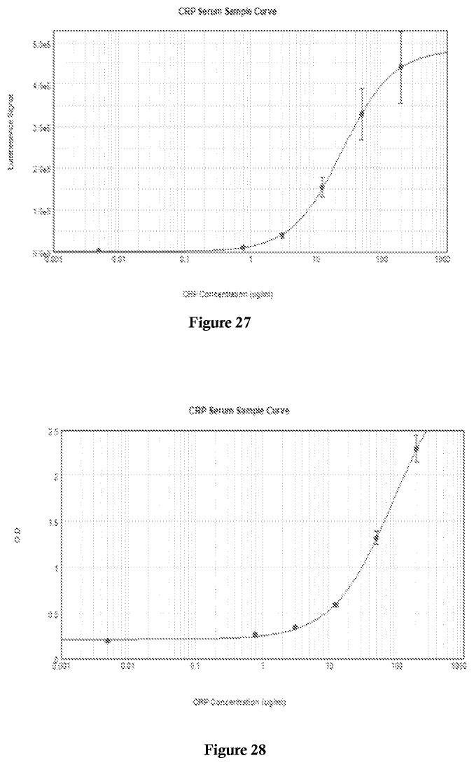

Table 13 below shows the Signal, % CV and % Maximum Signal for the CRP luminescent values and OD of calibration lines. The graphs in FIG. 27 and FIG. 28 show each line plotted individually as mean signal with standard deviation error bars. It can be seen that the beads of invention produce rather different shape of curve to the ELISA. The Covilyte line shows greater signal to noise but has a slightly increased variation at the highest CRP Concentrations.

### Luminescence Values

TABLE 13
Signal, % CV and % Maximum Signal for the CRP luminescent values

| Serum | | Final | | | | | | | | % |
|---|---|---|---|---|---|---|---|---|---|---|
| Calibrator | | Concentration | Day 0 21 Mar. 2017 | | Day 9 30 Mar. 2017 | | | | | Maximum |
| (ug/ml) | Dilution | (pg/ml) | well 1 | well 2 | well 1 | well 2 | Mean | StDev | % CV | Signal |
| 0.005 | 1000 | 5 | 2376 | 1812 | 3854 | 2945 | 2747 | 871 | 31.7 | 0.622 |
| 0.781 | 1000 | 781 | 8960 | 8928 | 12508 | 11417 | 10453 | 1798.8 | 17.2 | 2.37 |
| 3.125 | 1000 | 3125 | 31538 | 37524 | 39854 | 48380 | 39324 | 6980 | 17.7 | 8.90 |
| 12.5 | 1000 | 12500 | 142659 | 131595 | 183942 | 160388 | 154646 | 22849 | 14.8 | 35.0 |
| 50 | 1000 | 50000 | 362094 | 398552 | 280885 | 272792 | 328581 | 61661 | 18.8 | 74.4 |
| 200 | 1000 | 200000 | 566624 | 429363 | 373537 | 397620 | 441786 | 86308 | 19.5 | 100 |

| | | | | | |
|---|---|---|---|---|---|
| Signal to Noise | 238 | 237 | 97 | 135 | 161 |

### Serum Calibration Line— O.D.

TABLE 14
Signal, % CV and % Maximum Signal for the OD

| Serum | | Final | | | | | | % |
|---|---|---|---|---|---|---|---|---|
| Calibrator | | Concentration | 29 Mar. 2017 | | | | | Maximum |
| (ug/ml) | Dilution | (pg/ml) | well 1 | well 2 | Mean | StDev | % CV | Signal |
| 0.005 | 125000 | 0.04 | 0.182 | 0.198 | 0.190 | 0.0113 | 5.95 | 8.28 |
| 0.781 | 125000 | 6.25 | 0.276 | 0.237 | 0.257 | 0.0276 | 10.8 | 11.2 |
| 3.125 | 125000 | 25 | 0.354 | 0.332 | 0.343 | 0.0156 | 4.54 | 15.0 |
| 12.5 | 125000 | 100 | 0.573 | 0.597 | 0.585 | 0.0170 | 2.90 | 25.5 |
| 50 | 125000 | 400 | 1.264 | 1.37 | 1.32 | 0.0750 | 5.69 | 57.4 |
| 200 | 125000 | 1600 | 2.397 | 2.191 | 2.29 | 0.1457 | 6.35 | 100 |

| | | | |
|---|---|---|---|
| Signal to Noise | 13.2 | 11.1 | 12.1 |

Covilyte Calibration Line and ELISA Calibration Line are shown in FIGS. 27 and 28.

### Serum QC Sample Comparison

Table 15 below shows the signal and calculated concentration along with % CV and % Bias calculated for the serum quality control samples using the Covilyte and ELISA methods as well as the % difference between the concentrations calculated using the Covilyte and ELISA methods. The mean concentration with standard deviation error bars at each QC level for both methods are plotted on the three column charts. A correlation plot of Covilyte mean concentration vs ELISA mean concentration and plots comparing the % BIAS and % CV at each QC level for both methods are also shown.

It can be seen that the mean result and % CV are broadly comparable for both methods at all three QC levels although the Covilyte assay does show an increased % BIAS and % CV at the highest QC level an effect likely due to the flatter shape of the Covilyte calibration curve at the higher CRP concentrations.

TABLE 15
QCs Luminescence Signal using covilyte

| | | Analysed | 22 Mar. 2017 | | 23 Mar. 2017 | | 27 Mar. 2017 | | | | |
|---|---|---|---|---|---|---|---|---|---|---|---|
| Serum QC | | Concentration | Cartridge | Cartridge | Cartridge | Cartridge | Cartridge | Cartridge | | | |
| (ug/ml) | Dilution | (pg/ml) | 1 | 2 | 1 | 2 | 1 | 2 | Mean | StDev | % CV |
| 4 | 1000 | 4000 | 47567 | 49796 | 42416 | 36728 | 42693 | 41468 | 43444 | 4647 | 10.7 |
| 20 | 1000 | 20000 | 178235 | 174946 | 172695 | 159363 | 143526 | 152153 | 163486 | 13982 | 8.55 |
| 100 | 1000 | 100000 | 385220 | 386305 | 300018 | 374056 | 275290 | 421752 | 357107 | 56679 | 15.9 |

TABLE 16
QCs Calculated Concentration (μg/ml) using covilyte

| | | Analysed | 22 Mar. 2017 | | 23 Mar. 2017 | | 27 Mar. 2017 | | | | | |
|---|---|---|---|---|---|---|---|---|---|---|---|---|
| Serum QC | | Concentration | Cartridge | Cartridge | Cartridge | Cartridge | Cartridge | Cartridge | | | | |
| (ug/ml) | Dilution | (pg/ml) | 1 | 2 | 1 | 2 | 1 | 2 | Mean | StDev | % CV | % Bias |
| 4 | 1000 | 4000 | 3.46 | 3.61 | 3.08 | 2.67 | 3.10 | 3.01 | 3.15 | 0.337 | 10.7 | −21.2 |
| 20 | 1000 | 20000 | 15.6 | 15.2 | 14.9 | 13.5 | 11.7 | 12.6 | 13.9 | 1.56 | 11.2 | −30.4 |
| 100 | 1000 | 100000 | 86.4 | 96.9 | 47.5 | 92.9 | 32.3 | 139 | 82.6 | 38.2 | 46.3 | −17.4 |

Table 17 and 18; QCs Luminescence Signal and QCs Calculated concentration using ELISA
ELISA QCs O.D

| | | Analysed | | | | | | | | | |
|---|---|---|---|---|---|---|---|---|---|---|---|
| Serum QC | | Concentration | 29 Mar. 2017 | | | | | | | | |
| (ug/ml) | Dilution | (pg/ml) | Rep 1 | Rep 2 | Rep 3 | Rep 4 | Rep 5 | Rep 6 | Mean | StDev | % CV |
| 4 | 125000 | 32 | 0.283 | 0.297 | 0.290 | 0.326 | 0.327 | 0.354 | 0.312 | 0.0273 | 8.75 |
| 20 | 125000 | 160 | 0.736 | 0.739 | 0.689 | 0.762 | 0.751 | 0.714 | 0.732 | 0.0264 | 3.61 |
| 100 | 125000 | 800 | 1.84 | 1.84 | 1.79 | 1.85 | 1.88 | 1.90 | 1.85 | 0.0375 | 2.03 |

ELISA QCs Calculated Concentration (μg/ml)

| | | Analysed | | | | | | | | | | |
|---|---|---|---|---|---|---|---|---|---|---|---|---|
| Serum QC | | Concentration | 29 Mar. 17 | | | | | | | | | |
| (ug/ml) | Dilution | (pg/ml) | Rep 1 | Rep 2 | Rep 3 | Rep 4 | Rep 5 | Rep 6 | Mean | StDev | % CV | % Bias |
| 4 | 125000 | 32 | 2.412 | 2.765 | 2.580 | 3.483 | 3.512 | 4.260 | 3.17 | 0.706 | 22.3 | −20.8 |
| 20 | 125000 | 160 | 18.2 | 18.4 | 16.2 | 19.5 | 19.0 | 17.3 | 18.1 | 1.20 | 6.64 | −9.59 |
| 100 | 125000 | 800 | 108 | 109 | 103 | 110 | 114 | 118 | 110 | 5.17 | 4.69 | 10.2 |

### Conclusion

The assay in accordance with the proposed invention has been developed using the Covilyte (invention) system which can measure CRP in serum across the elevated range of 200-0.781 μg/ml with a total sample processing time below 15 minutes and additionally gives levels of accuracy and precision which are broadly comparable with those achieved using a conventional ELISA technique with total sample processing time of 6 hours. The Covilyte assay has shown acceptable stability for over a period of 9 days, though long term stability has not been assessed.Although a sample dilution step is required, but even that is 100 fold less than that required for a standard ELISA analysis.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations and for the detection of various analytes.

We claim:

1. An automated quantitative assay device comprising:
a) a means to receive a target sample collected from a subject; and
b) a means to measure and process a plurality of target analytes possibly present in the target sample in real time manner;
wherein:
said means to receive the target sample comprises a cassette device (100);
said means to measure and process the plurality of target analytes comprises a measuring instrument (200) having a plurality of means to assist the quantitative assay and an automated opening to allow the cassette device to be inserted within the measuring instrument; and
said automated quantitative assay device is capable of performing heterogeneous competitive or sandwich assays or immunological assays of the target sample across an elevated range in 10-15 minutes.

2. The automated quantitative assay device as claimed in claim 1, wherein the cassette device (100) comprises of:
a) a sample inlet (105);
b) a fluid conduit (102) extending between a sample inlet and a waste outlet (108) to a waste reservoir (104);
c) a mixing chamber (106) downstream but close to the sample inlet;
d) a plurality of measurement chambers disposed along the fluid conduit downstream of the mixing chamber and upstream of the waste outlet;
e) a reagent inlet port (107) downstream of the mixing chamber but before the first measurement chamber; and
f) a transparent cover (101);
wherein:
said fluid conduit contains a plurality of beads (103) fitted into a plurality of pockets in the fluid conduit so that the beads do not move with fluid;
said fluid conduit fluid is a continued channel fluidly connecting the inlet port, the mixing chamber, and the reagent port;
said waste reservoir (104) receives extra fluid through the waste outlet (108);
said transparent cover (101) allows recording of chemiluminescence.

3. The automated quantitative assay device as claimed in claim 2, wherein said beads (103) comprises of pre-labelled immobilized analyte-specific probe including but not limited to an antibody, aptamers or other affinity binding entities capable of being eroded by the reagent or the sample fluid used in the assay.

4. The automated quantitative assay device as claimed in claim 2, wherein said beads (103) comprises of pre-labelled immobilized analyte-specific probe are placed individually in a plurality of wells of the cassette device which is sealed using permanent heat seals.

5. The automated quantitative assay device as claimed in claim 2, wherein the mixing chamber is filled with a predetermined amount of a cocktail of conjugate molecules tagged with a chemiluminescent molecule, facilitating a competitive assay measurement wherein said conjugate molecules in the cocktail of conjugate molecules have an individual predetermined concentration.

6. The automated quantitative assay device as claimed in claim 2, wherein the mixing chamber is filled with a predetermined amount of the cocktail of conjugate molecules tagged with a chemical label that binds specifically to the analyte specific probes tagged with the chemiluminescent molecule, facilitating a sandwich assay measurement wherein each analyte specific probe in the cocktail of conjugate molecules has an individual predetermined concentration.

7. The automated quantitative assay device as claimed in claim 2, wherein said cocktail of conjugate molecules is pre-packaged in the cassette device in solid form which is formulated to dissolve to form a reagent fluid.

8. The automated quantitative assay device as claimed in claim 2, wherein a mixture of a cocktail of conjugate molecules and a cocktail of analyte specific probe molecules each tagged with a chemiluminescent molecule are possibly mixed in the same mixing chamber to configure the cassette device for an assay and a confirmatory.

9. The automated quantitative assay device as claimed in claim 1, wherein the measuring instrument (200) comprises of:
a) a closed housing (203) to prevent the enclosure from external light;
b) a plurality of reservoirs (201) containing predefined reagents;
c) at least 3 pumps (204) to pump reagents to the inlet ports (107) on the cassette device (100);
d) an optical sensor (206) that scans the cassette device (100) for detecting chemiluminescence;
e) a heating element (205) to heat the fluid conduit to 37° C.;
f) a stirring mechanism to mix the contents of the mixing chamber properly; said stirring mechanism includes but is not limited to magnetic stirrer;
g) an automated opening to allow the cassette device to be inserted; and
h) an electronic controller (209) to communicate with the external devices via wi-fi or bluetooth or wired communication;
wherein:
said optical sensor (206) is located by the use of a main gear (301) driven by either a stepper or geared direct current motor;
said optical sensor is either a photomultiplier tube or a multi pixel photon counting detector, or a silicone photomultiplier;
said heating element (205) is further associated with a cartridge heater (304) to heat the temperature set at 37° C. during the heating process which is monitored by a temperature sensor (304);
said pumps (204) pump reagents to the inlet ports (107) via an injection needle (208) through the silicone rubber cap (109); and
said inlet ports (107) are at the bottom of the cassette (100).

10. The automated quantitative assay device as claimed in claim 9, wherein said reservoirs contain wash and reagents required to trigger the chemiluminescence reaction.

11. The automated quantitative assay device as claimed in claim 9, wherein the optical sensor is a modified silicon photomultiplier comprising of:
a) a fan (401);
b) a heat sink (402);
c) a peltier (403);
d) a sensor board (404) enclosed in an enclosure (405) and a glass window (406) to protect condensation;
wherein,
said modified silicon photomultiplier achieves adequate signal to noise ratio by reducing dark current; and
said enclosure and glass window protect the sensor board from condensation.

12. The automated quantitative assay device as claimed in claim 9, wherein the detector is placed directly above each of the plurality of wells sequentially where geared direct current motors are used to rotate the light sensor into position where if the fluid conduit forms a circular path on the cassette device, the direct current motor is moved in a circular path around a fulcrum.

13. The automated quantitative assay device as claimed in claim 9, wherein the light sensor moves in a rectilinear fashion when the beads are placed in a grid pattern.

14. The automated quantitative assay device as claimed in claim 9, wherein the heating element is an infrared LED of wavelength 1500 nm or a radiative infrared emitter.

15. The automated quantitative assay device as claimed in claim 9, wherein the temperature sensor (304) is a non-contact thermometer.

16. The automated quantitative assay device as claimed in claim 9, wherein heating element (205) along with the injection needles (208) move as a unit to engage with the cassette device.

17. A method of automated quantitative assay comprising the steps of:
a) obtaining a cassette device fully loaded with a plurality of beads and an antibody/conjugate cocktail;
b) injecting a sample into a port at or upstream of a mixing chamber and filling the mixing chamber, the sample may be blood, saliva, serum, and urine;
c) inserting the cassette device into a measuring instrument;
d) activating a magnetic stirrer to mix the sample and the conjugate/antibody cocktail;
e) pumping a neutral liquid or air into the mixing chamber causing the sample/conjugate/antibody mixture to be displaced and to flow round a fluid conduit immersing the beads;
f) heating a liquid to approximately 37° C. to allow incubation;
g) pumping the wash through the fluid conduit to remove all of the remaining sample not bound to the beads;
h) repeating the wash cycles and purging the fluid conduit of the liquid by pumping air through the conduit between the wash cycles;
i) measuring the chemiluminescence through a sensor and recording a result;
j) ejecting the cassette device from the measuring instrument; and
k) shutting down the device.

18. The assay method as claimed in claim 17, wherein the magnetic stirrer is activated by placing a motor comprising a magnet attached to shaft below the mixing chamber and wherein the magnet and a shaft are rotated by the motor thereby resulting in the rotation of the small magnet in the mixing chamber.

19. The device and method as claimed in claims 1 and 17, wherein said assay is used for the detection of novel coronavirus 2019-nCoV, SARS-CoV2 or viral or bacterial outbreak, antigens; antibodies, particularly those induced in response to an infection, allergic reaction, or vaccine; hormones, proteins and other physiological substances for example, human chorionic gonadotropin, estrogens, progestins, testosterones, corticosteroids, human growth factors, hemoglobin, and cholesterol; nucleic acids; a variety of enzymes; therapeutic compounds and illicit drugs;
contaminants and environmental pollutants; or any number of natural or synthetic substances; ACE inhibitors, adrenergics and anti-adrenergics, alcohol deterrents for example, disulfiram, anti-allergics, anti-anginals, anti-arthritics, anti-infectives including antibacterials, antibiotics, antifungals, antihelminthics, antimalarials and antiviral agents, analgesics and analgesic combinations, local and systemic anesthetics, appetite suppressants, antioxidants, anxiolytics, anorexics, antiarthritics, anti-asthmatic agents, anticoagulants, anticonvulsants, antidiabetic agents, antidiarrheals, anti-emetics, anti-epileptics, antihistamines, anti-inflammatory agents, antihypertensives, antimigraines, antinauseants, antineoplastics, antioxidants, antiparkinsonism drugs, antipruritics, antipyretics, antirheumatics, antispasmodics, antitussives, adrenergic receptor agonists and antagonists, anorexics, appetite suppressants, cardiovascular preparations including anti-arrhythmic agents, cardiotonics, cardiac depressants, calcium channel blockers and beta blockers), cholinergics and anticholinergics, contraceptives, diuretics, decongestants, growth stimulants, herbal preparations, hypnotics, immunizing agents, immunomodulators, immunosuppressives, muscle relaxants, neurologically-active agents including anti-anxiety preparations, antidepressants, antipsycotics, psychostimulants, sedatives and tranquilizers, sore throat medicaments, sympathomimetics, vasodilators, vasoconstrictors, vitamins, xanthine derivatives, various combinations of these compounds, and the like.
